SEQUENCE LISTING (7 sequences)

SEQ ID NO: 1 - protein, 60 aa, Staphylococcus aureus
KKEQKLIQAQNLVREFEKTHTVSAHRKAQKAVNLVSFEYKVKKMVLQERIDNVLKQGLVR

SEQ ID NO: 2 - protein, 57 aa, Staphylococcus aureus
KKVVDAQKAVNLFKRTRTVATHRKAQRAVNLIHFQHSYEKKKLQRQIDLVLKYNTLK

SEQ ID NO: 3 - protein, 137 aa, Staphylococcus aureus
ASEGYGPREKKPVSINHNIVEYNDGTFKYQSRPKFNSTPKYIKFKHDYNILEFNDGTFEYGARPQFNKPAAKTDATIKKEQKLIQAQNLVREFEKTHTVSAHRKAQKAVNLVSFEYKVKKMVLQERIDNVLKQGLVR

SEQ ID NO: 4 - protein, 72 aa, Staphylococcus aureus
FNKPAAKTDATIKKEQKLIQAQNLVREFEKTHTVSAHRKAQKAVNLVSFEYKVKKMVLQERIDNVLKQGLVR

SEQ ID NO: 5 - protein, 80 aa, Staphylococcus aureus
QTKNVEAAKKYDQYQTNFKKQVNKKVVDAQKAVNLFKRTRTVATHRKAQRAVNLIHFQHSYEKKKLQRQIDLVLKYNTLK

SEQ ID NO: 6 - protein, 69 aa, Staphylococcus aureus
DQYQTNFKKQVNKKVVDAQKAVNLFKRTRTVATHRKAQRAVNLIHFQHSYEKKKLQRQIDLVLKYNTLK

SEQ ID NO: 7 - protein, 21 aa, Homo sapiens
SVQLTEKRMDKVGKYPKELRK

(19) United States
(12) Patent Application Publication
(10) Pub. No.: US 2011/0046075 A1
(43) Pub. Date: Feb. 24, 2011

(54) Secreted Staphylococcus Aureus Proteins And Peptides For Use In Inhibiting Activation Of The Complement System

(76) Inventors: John D. Lambris, Bryn Mawr, PA (US); Brian V. Geisbrecht, Lees Summit, MO (US)

Correspondence Address: POTTER ANDERSON & CORROON LLP;ATTN: JANET E. REED, PH.D., P.O. BOX 951, WILMINGTON, DE, 19899-0951, US

(21) Appl. No.: 12/526,222

(22) PCT Filed: Feb. 6, 2008
(86) PCT No.: PCT/US08/01662

§ 371 Date: Nov. 8, 2010

Related U.S. Application Data
(60) Provisional application No. 60/900,004, filed on Feb. 7, 2007.

(51) Int. Cl. A61K 38/16; C07K 14/31; C07H 21/00; C07K 16/12; C07K 16/18; C07K 14/00; A61P 37/02; A61P 29/00

(52) U.S. Cl. 514/21.3; 530/324; 536/23.7; 530/387.9; 530/389.1; 530/402

(57) ABSTRACT

Compounds comprising peptides and peptide analogs capable of binding the C3 protein and inhibiting complement activation are disclosed. These compounds mimic the structure and activity of secreted Staphylococcus aureus proteins, Efb and the previously uncharacterized SAV1 155.

### FIELD OF THE INVENTION

This invention relates to activation of the complement cascade in the body. In particular, this invention provides proteins and peptides capable of binding the C3 protein and inhibiting complement activation.

### BACKGROUND OF THE INVENTION

Various publications, including patents, published applications, technical articles and scholarly articles are cited throughout the specification. Each of these cited publications is incorporated by reference herein, in its entirety. Full citations for publications not cited fully within the specification are set forth at the end of the specification.

The complement system is the first line of immunological defense against foreign pathogens. Its activation through the classical, alternative or lectin pathways leads to the generation of anaphylatoxic peptides C3a and C5a and formation of the C5b-9 membrane attack complex. Complement component C3 plays a central role in activation of all three pathways. Activation of C3 by complement pathway C3 convertases and its subsequent attachment to target surface leads to assembly of the membrane attack complex and ultimately to damage or lysis of the target cells. C3 is unique in that it possesses a rich architecture that provides a multiplicity of diverse ligand binding sites that are important in immune surveillance and immune response pathways.

Inappropriate activation of complement may lead to host cell damage. Complement is implicated in several disease states, including various autoimmune diseases, and has been found to contribute to other clinical conditions such as adult respiratory syndrome, heart attack, rejection following xenotransplantation and burn injuries. Complement-mediated tissue injury has also been found to result from bioincompatibility situations such as those encountered in patients undergoing dialysis or cardiopulmonary bypass.

Complement-mediated tissue injuries are directly mediated by the membrane attack complex, and indirectly by the generation of C3a and C5a. These peptides induce damage through their effects on various cells, including neutrophils and mast cells. Under normal physiological conditions, activation of the complement system is strictly regulated by a series of proteins termed Regulators of Complement Activation (RCA). Among these are soluble serum proteins, such as factor H (1H) and C4b-binding protein (C4BP), as well as the membrane-bound proteins complement receptor one (CR1, CD35), membrane cofactor protein (MCP, CD46) and decay accelerating factor (DAF, CD55). In general, RCA proteins act at the level of C3 and C4 and function by dissociating the subunits of C3 and/or C5 convertases or by acting as a cofactors for the factor I-dependent cleavage of C3b and/or C4b.

To date, there are no inhibitors of complement activation approved for use in the clinic, though certain candidates for clinical use exist, specifically, a recombinant form of complement receptor 1 known as soluble complement receptor 1 (sCR1) and a humanized monoclonal anti-05 antibody (5G1.1-scFv). Both of these substances have been shown to suppress complement activation in in vivo animal models. However, each substance possesses the disadvantage of being a large molecular weight protein (240 kDa and 26 kDa, respectively) that is difficult to manufacture and must be administered by infusion. Accordingly, recent research has emphasized the development of smaller active agents that are easier to deliver, more stable and less costly to manufacture.

Staphylococcus aureus is a persistent human pathogen that is responsible for a wide-range of diseases that vary in both clinical presentation and severity. Perhaps more so than any other bacterial pathogen, S. aureus has evolved the ability to adapt to distinct microenvironments within the human body. Although the diverse functionality of its surface-bound adhesins (collectively termed MSCRAMMs for Microbial Surface Components Recognizing Adhesive Matrix Molecules²) clearly contributes to this ability, current models also suggest that the success of this organism as a pathogen is predicated on its capacity to manipulate and evade multiple host immune responses³⁻⁷. The circulating complement system is a primary target of virulence factors produced by many pathogens⁸. Due to its central role in activating all three complement pathways, complement component 3 (C3) represents a particularly attractive target for inhibition or modulation of the essential complement response^{5,9.} S. aureus has been shown to stimulate all three pathways of the complement system¹⁰⁻¹⁴, and mice that were complement-depleted by treatment with cobra venom factor (CVF) were found to be more susceptible to S. aureus-induced septicemia¹⁵. These results indicate that complement serves a principal role in the global immune response against S. aureus infection and suggest that the bacterium is likely to produce complement inhibitors that mimic the role of RCA proteins⁵. Indeed, conditioned S. aureus culture medium contains at least one C3b-binding protein, which was identified as the 15.6 lcDa extracellular fibrinogen-binding protein (Efb)⁵. Functional analysis revealed that recombinant Efb binds to the thioester-containing C3d-domain of C3b and inhibits C3b deposition onto sensitized surfaces^{5,6}, and that S. aureus strains incapable of expressing Efb were less virulent in a mouse model of wound healing¹⁶. Thus, attenuation of the complement response by S. aureus is a mechanism of immunosuppression that is advantageous to its role as a human pathogen.

### SUMMARY OF THE INVENTION

One aspect of the invention peptide features peptide components derived from secreted Staphylococcus aureus polypeptides, comprising a sequence selected from:

KKEQKLIQAQNLVREFEKTHTVSAHRKAQKAVNLVSFEYKVKKMVLQERID NVLKQGLVR (SEQ ID NO:1), or

KKVVDAQKAVNLFKRTRTVATHRKAQRAVNLIHFQHSYEKKKLQRQIDLVLKYNT LK (SEQ ID NO:2); wherein the peptide has a folded structure comprising, in order from the N- to C-terminus, a first αhelix of about 20 residues, a second αhelix of about 13 residues, and a third αhelix of about 17 residues, and comprising a random coil conformation of about 4 residues at its C-terminus, wherein the first and third αhelices are substantially parallel and the second αhelix is substantially anti-parallel to the first and third αhelices, and wherein the peptide binds to C3, induces a conformational change in C3, and inhibits C3-mediated complement pathway activation.

In one embodiment, the peptide comprises a structure substantially the same as a structure of a peptide shown in FIG. 1 for a C-terminal portion of Efb. In another embodiment, the peptide comprises a structure substantially the same as a structure of a peptide as shown in FIG. 2, for a C-terminal portion of Efb, bound to C3. In certain embodiments, the peptide is a C-terminal portion of Efb, or is contained within the S. aureus protein SAV1155, referred to herein as Ehp.

Another aspect of the invention features analog of the above-described peptide that retains the structure and function of the peptide, optionally comprising one or more of (1) naturally-occurring amino acids, (2) non-naturally-occurring amino acids, or (3) compounds that are not amino acids.

Also featured in the invention are polynucleotides that encode the above-described peptides. Another aspect of the invention features a variety of antibodies. In certain embodiments, the antibodies are immunologically specific for the peptides or analogs described above. In other embodiments, the antibodies are immunologically specific for the peptide binding regions on C3.

Another aspect of the invention features a method of inhibiting complement activation, comprising contacting a complement-forming system with the above-described peptide, under conditions permitting binding of the peptide to C3, resulting in the inhibition of the complement activation. In certain embodiments, the complement-forming system is contained within a living organism, such as a human or animal patient or subject.

Another aspect of the invention features a complex comprising C3 and a polypeptide secreted from Staphylococcus aureus, comprising SEQ ID NO:2, wherein the C3 within the complex is incapable of activating a complement pathway. In one embodiment, the polypeptide is SAV1155, GenBank Accession Number NP 371679. A method of inhibiting complement activation utilizing this polypeptide or portions thereof is also provided in accordance with the invention.

Other features and advantages of the present invention will be understood by reference to the detailed description, drawings and examples that follow.

### BRIEF DESCRIPTION OF THE DRAWINGS

FIG. 1. Crystal Structure of Efb-C at 1.25 Å. (A) Two orthogonal views of the refined Efb-C structure are shown in ribbon representation and colored according to the B factor where blue ˜28 and red ˜10 Å², respectively. (B) Surface charge distribution is displayed as blue for 15 electrons/Å and red for −15 electrons/Å. Positions of surface exposed basic residues are highlighted.

FIG. 2. Crystal Structure of the Efb-C/C3d Complex at 2.2 Å. (A) Two orthogonal views of the complex structure are shown. The C3d molecule in the schematic representation is colored from red at its N-terminus to the blue at its C-terminus; helices H1 to H12 are labeled. The Efb-C molecule is shown in ribbon representation and colored according to the refined B factor at each position, where blue ˜85 and green ˜40 Å², respectively; helices α1, α2 and α3 are labeled. Contacting residues R131 and N138 from Efb-C are highlighted in blue. (B) Stereo view of the Efb-C (blue) and C3d (red) interface. Residues involved in protein-protein contacts are labeled and shown as stick models. Two structural water molecules are involved in the R131 contact and are represented as silver spheres.

FIG. 3. Inhibition of C3 Activation by Efb Requires Binding to the C3d Domain. (A) The thermodynamics of the Efb/C3d interaction were examined quantitatively using an isothermal titration calorimetry (ITC) assay and site-directed mutagenesis. Fitted ITC binding curves are shown for titrations of Efb (black squares), Efb-N (blue diamonds), Efb-C (red circles), and R131A_N138A (orange triangles) and R131E_N138E double mutants (green triangles) of Efb-C into a solution of recombinant C3d. Legends and experimental values for equilibrium dissociation constants (K_{d}) and enthalpy (ΔH) are shown inset. (B) Concentration-dependent inhibition of the alternative pathway of complement activation was assessed for Efb (black squares), Efb-C (red circles), and the R131E_N138E double mutant (green triangles).

FIG. 4. Functional Basis for Complement Inhibition by Efb-C. (A) Conformational changes of plasma C3 were monitored using a capture ELISA. Diluted plasma containing EDTA (for blocking the complement cascade) was incubated with Efb-C, Efb-C-(RENE), or buffer alone for one hour at 37° C. and captured on mAb C3-9 coated to ELISA plates. Bound C3 was detected using an anti-C3a mAb and a HRP-conjugated goat-anti-rabbit Ab (HRP-GaR). The legend along with a cartoon representation of the assay is shown inset. (B) The change in C3 conformation was further investigated using a protease sensitivity assay. Purified C3 was incubated with the same proteins and subjected to limiting proteolysis by trypsin (0.1% w/w). Aliquots from each reaction were removed at 5, 10, 15, and 20 minutes and compared to a control sample of C3 lacking trypsin by reducing 9% SDS-PAGE and Coomassie staining. The contrast of the figure was increased uniformly to enhance visualization of the bands.

FIG. 5. Efb-C Binds to Native Forms of C3. (A) Results from a surface plasmon resonance (SPR) ranking experiment. Efb-C was immobilized on the surface of a sensor chip and various C3 intermediates were injected at a constant concentration of 200 nM. Each binding curve was normalized by dividing the SPR signal by the molecular weight of the injected protein, since the SPR response is dependent on the analyte mass. (B) SPR kinetic profiles of selected C3 fragments binding to Efb-C. The processed binding curves for each fragment are shown in black, and were fitted to kinetic models (orange curves) that describe either 1:1 Langmuir binding (C3(H₂O), C3b, and C3dg) or surface heterogeneity (C3 native). Kinetic rate constants derived from the results shown in panel B are presented in Table 2.

FIG. 6. Proposed Structural Basis for the Differential Recognition of C3 Fragments by Efb-C. (A) Localization of Efb-C in the context of native C3 based upon the Efb-C/C3d (FIG. 2) and C3 crystal structures¹⁸. The model is presented with C3 in the same orientation as shown in FIG. 1A of Janssen et al.¹⁸. Efb-C is drawn as a red surface (to avoid confusion with the ANA/C3a domain), while the C3d domain is shown as green tubes and the MG2 domain in orange. C3 peptides with alteied conformations during activation to C3(H₂O) are colored yellow. (A, Inset) A magnified view of the boxed region is shown with Efb-C as a red ribbon and several important sidechains presented as stick models. Note the proximity of Efb-C helix α3 to the MG2 domain of native C3. (B) Localization of Efb-C in the context of activated C3b based upon FIG. 2 above and the C3b crystal structure¹⁹. Efb-C is shown in this model as a red ribbon. (B, Inset Top) A magnified view of the boxed region is shown as in Panel A. The color scheme shown for C3 and C3b is adapted from Janssen et al. for the sake of clarityl^{18, 19}. (B, Inset Bottom) Efb-C binding induces a conformational change in C3b. SPR sensorgrams for binding of various analytes to immobilized monoclonal antibody C3-9 are shown as follows: C3b alone (solid blue trace), Efb-C/C3b theoretical response (dashed blue trace), Efb-C/C3b observed response (purple trace), and Efb-C or buffer alone (red and grey traces).

FIG. 7. Structural Comparisons Between Efb-C and Protein A Modules from S. aureus. (A) Ribbon representation of Efb-C (left panel) and the averaged solution NMR structure of a Protein A module from S. aureus (RSCB Accession Code 1BDD). Proteins are shown with an indexed color scheme, where the N-terminus appears in red and the C-terminus in blue. The locations of individual helices are indicated. (B) Schematic diagrams showing the topological relationships of individual helices in Efb-C (left panel) and Protein A modules (right panel).

FIG. 8. Spatial Relationship of Efb-C to the Conserved Acidic Groove of C3d as Seen in the Efb-C/C3d Crystal Structure. Efb-C is shown as a blue ribbon diagram, and positively charged residues that contact C3d are drawn in teal. C3d appears in its surface representation, where the predominantly acidic, conserved residues that form a negatively charged groove on the C3d surface are shown in red¹⁷. Efb-C residue positions appear in blue, while C3d residue positions are indicated in yellow.

FIG. 9. Structural Integrity of the Efb-C-(RENE) Mutant. Circular dichroism spectropolarimtery was used to compare the structural integrity of Efb-C-(RENE) (green trace) with wild-type Efb-C (red trace). The minima observed near 208 and 222 nm in both spectra are characteristic of the α-helical fold adopted by Efb-C.

FIG. 10. Efb-binding Renders C3 Susceptible to Proteolysis In Vitro. Control digests were performed to ensure the integrity of the proteolysis experiment presented in FIG. 4 as follows: (1) C3+trypsin+SBTI, (2) C3b+Efb-C, (3) C3b+Efb-C-(RENE), (4) C3b+trypsin+SBTI, (5) C3+Efb-C, and (6) C3+Efb-C-(RENE). The lack of digestion in the absence of trypsin indicates that there is no proteolytic activity intrinsic to the Efb-C protein itself.

FIG. 11. Degradation of C3 in Complement-inactivated Plasma in the Presence of Efb-C. The fate of C3 in complement-inactivated plasma was monitored by western blot analysis. Diluted EDTA plasma containing either Efb-C, Efb-C-(RENE), or no addition was incubated for two hours at 37° C. All samples were separated by reducing 9% SDS-PAGE and transferred to PVDF membranes. Immunoblots were probed with either a rabbit polyclonal anti-C3a or a rabbit polyclonal anti-C3b antibody (top panels) and the reactive bands were compared to purified C3, C3b, iC3b, and to CVF-treated lepirudin plasma. In both blots, additional bands were visible in case of Efb-C-treated plasma. As visualized in the bottom cartoon, the occurrence of a 77 kDa band in the anti-C3a and a 43 kDa band in the anti-C3b blot is indicative of a factor I (fI)-mediated cleavage with removal of the C3f fragment. In contrast, C3a has not been removed as is typically seen during normal degradation of C3 during complement activation. These results indicate that the fI cleavage sites are more exposed in the Efb-C/C3 complex than in native C3, even without cleavage to C3b. However, the intensity of the newly formed bands and the time required for the generation of these bands suggests that this seems not to be the major mechanism of complement inhibition but rather another indication of a conformational change of C3 upon Efb-C binding and the process by which bound C3 is ultimately degraded. Again as it was seen in the purified system (FIG. 4B), the double mutant Efb-C-(RENE) failed to produce the same degradation pattern seen with the wild type Efb-C. The anti-C3a antibody was affinity-purified using an immobilized N-terminal C3a peptide (indicated by a red bar in the bottom cartoon). The visualization of a 72 kDa band in all plasma samples may be derived from a cross-reactivity of anti-C3a with a protein of unknown nature in the plasma.

FIG. 12. Alternative Depiction of Structural Models for Efb-C Bound to C3 and C3b. The three dimensional coordinates of C3b¹⁹ were superimposed onto those of native C3¹⁸ by Local-Global Alignment³⁷, and an analogous images to those shown in FIG. 6 were prepared. The identity of each domain and/or protein is indicated. This view shows the substantial conformational changes associated with activation of native C3 to C3b, and the relative location of the Efb-C binding site in each structure.

FIG. 13. Graph and immunoblot showing that Ehp is expressed at highest levels during log-phase growth. Conditioned medium from S. aureus cultures was collected, normalized for cell density, and analyzed by immunoblotting for the presence of Ehp. Positive controls of purified, recombinant Ehp protein (+) and growth medium alone (−) are indicated. A slight increase in apparent molecular weight is due to the presence of an affinity tag.

FIG. 14. Structure-based sequence alignment of Efb and Ehp. Secondary structure elements are shown above the corresponding residues according to the legend: black dots (not observed), red tubes (alpha helix), and red lines (linker/non-classical).

FIG. 15. Ehp is a novel C3-binding protein. A 1:100 dilution of human serum was incubated with sepharose resin derivatized with either Ehp, Efb, Efb-C or control. Following separation by non-reducing SDS-PAGE, each isolated protein was identified by LC-MS/MS.

FIG. 16. Inhibition of complement pathway activation by Efb, Efb-C and Ehp. Data are shown for inhibition of complement activation through the classical pathway as measured by an ELISA-based method for C3b formation. Legend and IC50 values are inset.

FIG. 17. Isothermal titration calorimetry binding curves for Efb, Efb-C and Ehp to recombinant C3d. Binding constants for each interaction are inset. Note the presence of a second C3d binding site in Ehp.

FIG. 18. Schematic approach for the optimization of complement inhibitory molecules derived from the Efb family. In the top panel, the Ehp structure is depicted with all contiguous residues that define both C3-binding sites as stick representations. Residues that are found (for the “Efb-like” site), or presumed to be located (for the lower-affinity site) at the interface of the C3 complex are labeled. In the bottom panel, a stick model is shown for a hypothetical peptide derived from the Ehp protein. This molecule consists of the 29 contiguous residues described in the top panel, but also contains a series of mutations (labeled) predicted to convert the low-affinity site (top of the drawing) into one with properties more in-line with the higher-affinity sites (bottom of the drawing). C3-contacting residues are drawn in black.

### DETAILED DESCRIPTION OF ILLUSTRATIVE EMBODIMENTS

In accordance with one aspect of the invention, x-ray crystallography has been used to describe the three dimensional structure of the complement component 3 (C3) binding domain of the Extracellular Fibrinogen-binding Protein (Efb) that is secreted by Staphylococcus aureus. In addition, the inventors have solved the crystal structure of this portion of Efb (also denoted Efb-C) bound to its cognate domain, C3d, from human C3. This structure has identified two residues, R131 and N138, which are needed to form and maintain the Efb-C/C3d complex. Binding of C3 by S. aureus Efb-C induces a stable conformational change in C3 that renders this central complement component unable to participate in downstream activation of the complement immune response, and thereby prevents C3b generation.

In accordance with another aspect of the invention, a second S. aureus protein, denoted Ehp (for Efb Homologous Protein), was identified through a genomic scan for orphan proteins related to Efb. Ehp is 44% identical to the C3-inhibitory Efb-C domain, and multiple sequence alignments revealed that Ehp contains two residues, R75 and N82, that are analogous to Efb-C R131 and N138. Like Efb-C, Ehp contains a potent complement inhibitory activity that is predicated upon its ability to bind tightly to C3d. Disruption of this interaction by mutation of R75 and/or N82 results in a loss of inhibition. The similar structure and mechanism used by both Efb and Ehp indicates that the domain they share has been evolutionarily optimized to bind C3 and block activation of the complement response.

The potency of C3 inhibition by Efb-C and Ehp indicates that these molecules will be useful for the therapeutic inhibition of the complement system. These therapeutic uses may involve administration of recombinant and/or optimized forms of Efb-C and Ehp or, alternatively, administration of peptide fragments, mimetics, or small molecules designed to replicate the anti-complement activity of these proteins in vivo. The inventors have developed protocols to successfully prepare shortened forms of both Efb and Ehp that retain the complement inhibitory properties of the native proteins in addition to their excellent solubility characteristics. An example of one such protocol is set forth in Example 4.

Studies using specific complement inhibitors or complement knock-out animals have revealed that complement plays a crucial role in the pathogenesis of tissue inflammation in a number of animal disease models. These include local and remote damage after ischemia reperfusion, immune-complex and autoimmune disease. In addition, complement activation is known to be an aggravating factor in joint, kidney, and central nervous system diseases, Acute Respiratory Distress Syndrome, systemic inflammatory response due to sepsis or extracorporeal circulation, antibody-induced fetal loss, and allo- and xenotransplant graft rejections. As a consequence, therapeutic inhibition of the complement system has been suggested as an attractive approach to treat a number of these diseases and conditions.

In addition, Efb has an established role in promoting S. aureus virulence in a rodent model of wound infection and antibody-mediated disruption of Efb function has already been shown to be therapeutically valuable. Because the complement inhibitory properties of Efb appear to contribute to the ability of this bacterium to initiate serious and life-threatening infections, any small molecules, peptides or mimetics that block binding to C3d by Efb can also be useful in development of antibacterial agents. Such molecules would be expected to act by augmenting normal human immunity by blocking the anti-inflammatory action of the bacterial protein(s).

The polypeptides, peptides and analogs of the present invention may be prepared by various synthetic methods of peptide synthesis via condensation of one or more amino acid residues, in accordance with conventional peptide synthesis methods. For example, peptides are synthesized according to standard solid-phase methodologies, such as may be performed on an Applied Biosystems Model 431A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art. During the course of peptide synthesis, branched chain amino and carboxyl groups may be protected/deprotected as needed, using commonly-known protecting groups. Modification utilizing alternative protecting groups for peptides and peptide derivatives will be apparent to those of skill in the art.

Alternatively, polypeptides and peptides of the invention may be produced by expression in a suitable prokaryotic or eukaryotic system. For example, a DNA construct may be inserted into a plasmid vector adapted for expression in a bacterial cell (such as E. coli) or a yeast cell (such as Saccharomyces cerevisiae), or into a baculovirus vector for expression in an insect cell or a viral vector for expression in a mammalian cell. Such vectors comprise the regulatory elements necessary for expression of the DNA in the host cell, positioned in such a manner as to permit expression of the DNA in the host cell. Such regulatory elements required for expression include promoter sequences, transcription initiation sequences and, optionally, enhancer sequences.

Polypeptides and peptides produced by gene expression in a recombinant procaryotic or eucaryotic system may be purified according to methods known in the art. In one embodiment, a commercially available expression/secretion system can be used, whereby the recombinant peptide is expressed and thereafter secreted from the host cell, to be easily purified from the surrounding medium.

A combination of gene expression and synthetic methods may also be utilized to produce certain peptides and analogs. For example, an analog can be produced by gene expression and thereafter subjected to one or more post-translational synthetic processes, e.g., to modify the N- or C-terminus or to otherwise constrain the conformation of the molecule.

The structure of Efb is described herein, and Ehp has been determined by sequence homology and predicted protein folding to have similar structural components. Once a particular desired conformation of a short peptide has been ascertained, methods for designing a peptide or peptidomimetic to fit that conformation are well known in the art. See, e.g., G. R. Marshall (1993), Tetrahedron, 49: 3547-3558; Hruby and Nikiforovich (1991), in Molecular Conformation and Biological Interactions, P. Balaram & S. Ramasehan, eds., Indian Acad. of Sci., Bangalore, PP. 429-455). Of particular relevance to the present invention, the design of peptide analogs may be further refined by considering the contribution of various side chains of amino acid residues, as discussed above (i.e., for the effect of functional groups or for steric considerations).

It will be appreciated by those of skill in the art that a peptide mimic may serve equally well as a peptide for the purpose of providing the specific backbone conformation and side chain functionalities required for binding to C3 and inhibiting complement activation. Accordingly, it is contemplated as being within the scope of the present invention to produce C3-binding, complement-inhibiting compounds through the use of either naturally-occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “peptidomimetic” or “isosteric mimetic,” to designate substitutions or derivations of the peptides of the invention, which possess the same backbone conformational features and/or other functionalities, so as to be sufficiently similar to the exemplified peptides to inhibit complement activation.

The use of peptidomimetics for the development of high-affinity peptide analogs is well known in the art (see, e.g., Zhao B, Helms L R, DesJarlais R L, Abdel-Meguid S S, and Wetzel R. (1995) A paradigm for drug discovery using a conformation from the crystal structure of a presentation scaffold. Nat. Struct. Biol. 2:1131-7; Beeley N. (1994) Peptidomimetics and small-molecule drug design: towards improved bioavailability and in vivo stability. Trends Biotechnol. 12:213-6; Hruby V J. (1993) Conformational and topographical considerations in the design of biologically active peptides. Biopolymers. 33:1073-82). Assuming rotational constraints similar to those of amino acid residues within a peptide, analogs comprising non-amino acid moieties may be analyzed, and their conformational motifs verified, by means of the Ramachandran plot (Hruby & Nikiforovich 1991, in Molecular Conformation and Biological Interactions, P. Balaram & S. Ramasehan, eds., Indian Acad. of Sci., Bangalore, PP. 429-455), among other known techniques.

The peptides and analogs of the present invention can be modified by the addition of polyethylene glycol (PEG) components to the peptide. As is well known in the art, PEGylation can increase the half-life of therapeutic peptides and proteins in vivo. In one embodiment, the PEG has an average molecular weight of about 1,000 to about 50,000. In another embodiment, the PEG has an average molecular weight of about 1,000 to about 20,000. In another embodiment, the PEG has an average molecular weight of about 1,000 to about 10,000. In an exemplary embodiment, the PEG has an average molecular weight of about 5,000. The polyethylene glycol may be a branched or straight chain, and preferably is a straight chain.

The peptides and analogs of the present invention can be covalently bonded to PEG via a linking group. Such methods are well known in the art. (Reviewed in Kozlowski A, Charles S A, and Harris J M. (2001) Development of pegylated interferons for the treatment of chronic hepatitis C. BioDrugs. 15:419-29; see also, Harris J M and Zalipsky S, eds. Poly(ethylene glycol), Chemistry and Biological Applications, ACS Symposium Series 680 (1997)). Non-limiting examples of acceptable linking groups include an ester group, an amide group, an imide group, a carbamate group, a carboxyl group, a hydroxyl group, a carbohydrate, a succinimide group (including without limitation, succinimidyl succinate (SS), succinimidyl propionate (SPA), succinimidyl carboxymethylate (SCM), succinimidyl succinamide (SSA) and N-hydroxy succinimide (NHS)), an epoxide group, an oxycarbonylimidazole group (including without limitation, carbonyldimidazole (CDI)), a nitro phenyl group (including without limitation, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. In certain embodiments, the linking group is a succinimide group. In one embodiment, the linking group is NHS. The peptides and analogs can alternatively be coupled directly to PEG (i.e., without a linking group) through an amino group, a sulfhydral group, a hydroxyl group or a carboxyl group.

The complement activation-inhibiting activity of the peptides, analogs, peptidomimetics and conjugates described herein may be tested by a variety of assays known in the art. In a preferred embodiment, the assay described in Example 1 is utilized. A non-exhaustive list of other assays is set forth in U.S. Pat. No. 6,319,897, including, but not limited to, (1) peptide binding to C3 and C3 fragments; (2) various hemolytic assays; (3) measurement of C3 convertase-mediated cleavage of C3; and (4) measurement of Factor B cleavage by Factor D.

For optimum therapeutic utility, the immunogenicity of the compositions of the invention should be minimized. Methods for reducing immunogenicity are familiar to the person of skill in the art. For example, targeting the central human histocompatibility system, HLA proteins and their binding motifs have been found to be predictive of how an individual might immunologically react to a foreign protein. One useful product utilizing such targets is ImmunoFilter®, an experimentally derived database of HLA-binding prediction matrices that is purported to cover at least 90% of the U.S. population (Xencor, Inc., Monrovia, Calif.). The tool comprises a software package in which a selected protein sequence is entered into the program and a full report of HLA epitope propensities across the entire protein is generated. Other methods of reducing immunogenicity will be apparent to the skilled artisan.

The following examples are provided to describe the invention in greater detail. They are intended to illustrate, not to limit, the invention.

### Example 1

### Structural Basis for a Mechanism of Complement Inhibition by Staphylococcus aureus

This example presents structures of a bacterial complement inhibitory protein both free and bound to its complement target. The 1.25 Å structure of the C3-inhibitory domain of Staphylococcus aureus Efb (Efb-C) reveals a novel helical motif involved in complement regulation, while the 2.2 Å structure of Efb-C bound to the C3d domain of human C3 provides insight into recognition of complement proteins by invading pathogens. Structure-function studies provide evidence for a new mode of complement inhibition wherein Efb-C binds to native C3 and alters the solution conformation of C3 in a manner rendering it unable to participate in successful downstream activation of the complement response.

### Methods:

Protein Expression and Preparation. A DNA fragment encoding the C-terminal C3-binding domain (residues 94-165) of Efb from S. aureus strain Mu50 was PCR-amplified from the full-length Efb expression vector pT7HMT-Efb and used to prepare recombinant Efb-C as described²⁵. Site-directed mutations encoding the R131E and N138E double mutant (Efb-C-(RENE)) were introduced into the pT7HMT-Efb-C expression vector by the two-step megaprimer method²⁵, and the structural integrity of the Efb-C-(RENE) double mutant was validated by circular dichroism spectropolarimetry (Supplementary FIG. 3B) according to a previously reported method²⁵.

A DNA fragment encoding residues 974 through 1265 of human C3 was PCR amplified to encode the C1010A mutation and subcloned into a modified form of pET28a which lacked all tags. This vector encodes the additional residues G-S—R—S-T at its N-terminus and the C1010A mutation is necessary to avoid the formation of the reactive thioester bond found in native C3⁹′¹⁷. Protein expression and purification for recombinant C3d was carried out according to the general protocol described by Nagar¹⁷, with the exception that microfluidization was used to lyse the induced cells²⁵.

To obtain the Efb-C/C3d complex for further structural analysis, purified C3d was incubated with Efb-C for 10 min at room temperature at a molar ratio of Efb-C:C3d 2:1. Unbound Efb-C was separated by gel filtration chromatography on a Superdex 26/60 column (GE Healthcare), and the purified, reconstituted complex was dialyzed against double-deionized water and concentrated by ultrafiltration to 12 mg/ml total protein (as determined by UV-absorption spectrophotometry).

For functional characterization and biosensor binding experiments, purified C3, C3(H₂O), C3b, and C3c were all prepared according to established protocols. Recombinant C3dg was expressed in E. coli based on a method described by Guthridge et al.²⁶. A pET30 vector containing a S-tag was used instead of a pET11b vector with T7 epitope tag, and no biotinylation signal peptide was included.

Crystallization, Structure Determination, and Analysis. The crystallization and X-ray diffraction analysis of Efb-C has been described previously²⁷. Following determination of the initial experimental phases to 2.2 Å from Selenium MAD data using the program SOLVE²⁸, final experimental phases at the same resolution were calculated through density modification protocols, and the resulting electron density maps were used for automated model building using the program RESOLVE²⁸⁻³⁰. An initial atomic model of both Efb-C chains was built into the density-modified 2.2 Å maps (Table 1) using the program O³¹, and was subjected to a single round of simulated annealing and maximum-likelihood positional refinement using CNS³² and a native data set processed to 1.25 Å limiting resolution²⁷. Iterative rounds of model building and water addition were alternated with B-factor and positional refinement, and the final model was attained by TLS refinement in using REFMAC5^{33,34}. Although the crystallographically unique unit is a dimer, this is not likely to reflect any physiologically relevant oligomerization since Efb-C is a monomer in solution. The final model consists of residues 105-165 in molecule A, 106-165 in molecule B, and 155 water molecules. No interpretable density is seen for residues 94-104 in molecule A or 95-105 in molecule B, and the conformation of the side chains of I105 and R165 in molecule A and I105, K106, and K107 in molecule B are not interpretable beyond the Cβ positions and were therefore modeled as alanine.

Crystals of Efb-C/C3d were grown by vapor diffusion of hanging drops where 1 μl of 12 mg/ml Efb-C/C3d was mixed with 1 μl of 60% tacsimate (pH 7.0) (Hampton Research) and equilibrated against a 60% tacsimate (pH 7.0) well solution (1 ml). Block-shaped single crystals grew within 3 days in space group P4₁ with cell dimensions of a=b=90.94 Å, c=120.24 Å and two complexes in the asymmetric unit, which corresponds to solvent content of 59%. The structure of Efb-C/C3d was solved by molecular replacement using X-ray diffraction data to 2.2 Å limiting resolution collected from a single frozen crystal at SER-CAT Beamline 22-BM of the Advanced Photon Source of Argonne National Laboratory. Following data processing using HKL2000 software package, molecular replacement was carried out using the refined structure of C3d as a search model (RCSB Accession Number 1C3D¹⁹) using the program MOLREP³⁵. Initial phase improvement was carried out using solvent flattening by the program DM³⁵, and stepwise model building, water addition and refinement were carried out using programs O³¹ and CNS³² as described above. The final model was attained following TLS refinement in REFMAC5³³′³⁴, and consists of the vector-encoded sequence GSRST in addition to residues 974-1265 of human C3 and residues 101-165 of Efb-C for both complexes in the asymmetric unit, as well as 281 ordered water molecules. Although the crystallographic unit is a dimer, this is not likely to reflect physiologically relevant oligomerization since the complex itself is monomeric in solution. No interpretable density for residues 94-100 is visible for either molecule of Efb-C in the refined model.

Structural Analysis. Analyses of molecular surfaces and electrostatic field potentials were performed and displayed using the program GRASP³⁶. Intramolecular distances for protein chains were calculated using the CCP4 suite³⁵. Structural superpositions of proteins were carried out by the Local Global Alignment method of Zemla using the default parameters³⁷ (available on the web at http://as2ts.11nl.gov/).

Isothermal Titration calorimetry. ITC experiments were performed at 25° C. using a VP-ITC (Microcal) calorimeter. All proteins were dissolved in 20 mM Tris (pH 8.0) and 200 mM NaCl to a final concentration of either 150 μM for S. aureus components or 12 μM for recombinant C3d. A preliminary 1 μl injection of the S. aureus protein into a solution of C3d was followed by multiple injections of 5 μl at 240 s intervals and the evolved heat was measured. All data were fit using Origin software (OriginLab) using a single binding site model to give the final experimental values for both enthalpy (ΔH) and entropy (-TΔS) of binding after correcting the observed heat values for the effect of dilution and subtraction of buffer titration baseline values.

Assays for Inhibition of Complement Pathway Activation. The ability of various Efb-derived proteins to inhibit the classical and alternative pathways of complement activation was evaluated using an ELISA-based approach originally described by Kraus³⁸ and as employed in Sfyroera et al.³⁹. Since the primary mode of Efb-mediated inhibition is on the alternative activation pathway (see Results), this assay was employed more extensively during this work. To assess the effects of Efb on the alternative pathway, ninety-six-well ELISA plates were coated with 40 μg/ml LPS from Salmonella typhosa in PBS for 2 h at room temperature. After coating, wells were blocked with 200 μl of 1% BSA for 1 h, and Efb, Efb-C, or its mutant was serially-diluted in GVB-MgEGTA (veronal-buffered saline, pH (7.4), 5 mM barbital, 145 mM NaCl, 0.1% gelatin, 0.1 M MgCl₂, and 0.1 M EGTA) and added to the wells. Human serum in the same buffer at a final concentration of 1/10 was then added to each well and incubated for 1 h. To detect C3b bound to the wells, a 1/1000 dilution of anti-human C3 HRP-conjugated Ab (ICN Cappel) was used. Following extensive washing with PBS containing 0.005% Tween 20, color was developed by adding ABTS peroxidase substrate, and OD was measured at 405 nm. Percent inhibition was plotted against the protein concentration and the resulting data set was fit to a logistic dose-response function using Origin 7.0 software (OriginLab Corporation). IC50 values were obtained from the fit parameters that achieved the lowest χ² value.

C3 Capture ELISA. A capture ELISA method was developed to test for conformational changes in C3 induced by binding to Efb-C. EDTA plasma diluted 1:10 in PBS was incubated with or without 25 μM Efb-C or Efb-C-(RENE) at 37° C. for 1 hour and centrifuged at 3.000 rpm for 5 min. The supernatant was added to an ELISA plate coated with the monoclonal antibody C3-9 (2 μg/ml), which recognizes a neoantigen that is exposed in C3(H₂O), C3b, and C3c but not in native C3. The wells were washed twice with PBS containing 0.005% Tween 20 and incubated with a rabbit polyclonal anti-C3a Ab (2 μg/ml) that had been affinity-purified on a CNBr-Sepharose column derivatized with an N-terminal C3a peptide (H-SVQLTEKRMDKVGKYPKELRK-CONH₂; SEQ ID NO:7). The final detection was performed using goat anti-rabbit IgG-HRP (1:1000) and ABTS peroxidase substrate by measuring the OD at 405 nm. Values were plotted against the protein concentration and the resulting data set was fit to a logistic-dose response function using Origin 7.0 (OriginLab).

Trypsin Sensitivity Assay. 10 μg of C3 were incubated at 37° C. with 0.1% (w/w) trypsin in the presence of 5.2 μM Efb-C, Efb-C-(RENE), or in buffer alone. Samples were collected at 0, 5, 10, 15 and 20 min following initiation of the reaction. Each reaction was quenched with soy bean trypsin inhibitor (SBTI) and the samples were reduced, denatured, and separated by electrophoresis through a 9% SDS-PAGE gel. Protein bands were visualized by Coomassie staining.

Plasma Degradation Assay. The fate of C3 in human plasma containing either Efb-C, Efb-C-(RENE), or buffer alone was monitored by immunoblotting. Briefly, plasma samples were pretreated with either EDTA (to block complement activation) or lepirudin and were incubated for 2 hours at 37° C. with stoichiometric concentrations of either Efb-C, Efb-C-(RENE), buffer alone, or cobra venom factor (CVF) in the case of lepirudin-treated plasma (as a positive control of complement activation). All samples were separated using 9% SDS-PAGE under reducing conditions and transferred to PVDF membranes. Following blocking with non-fat dry milk (10% (w/v) in PBS), the membrane was probed with either an affinity-purified polyclonal rabbit anti-C3a or a polyclonal rabbit anti-C3b antibody. Bound antibodies were detected by chemiluminescence following exposure to an HRP-conjugated goat anti-rabbit secondary antibody.

Surface Plasmon Resonance of Efb Binding to Components of the Complement System. All SPR experiments were performed on a Biacore X (ranking) or Biacore 2000 (kinetic profiling) biosensor at 25° C. using 10 mM PBS-T (10 mM sodium phosphate, pH 7.4, 150 mM NaCl, 0.005% Tween-20) as running buffer. The ranking assay was performed as essentially described by Sfyroera et al. ⁴⁶. Briefly, 600 resonance units (RU) of Efb-C were immobilized on a CM5 sensor chip using amine coupling according to the manufacturer's suggestions. A separate flow cell was only activated and deactivated, and served as reference surface. 200 nM solutions of the C3 fragments were injected for 3 min at a flow rate of 20 μl/min with a dissociation phase of 3 min. The Efb-C surface was regenerated with two consecutive 30 s pulses of 0.1% SDS and re-equilibrated in running buffer for 10 min. For the kinetic SPR studies, Efb-C was immobilized at lower density (200 RU) and the flow rate was kept at 30 μl/ml in order to avoid mass transport effects. C3 fragments were screened as threefold dilution series (67-0.09 nM; 200-0.09 nM for C3b) in running buffer with injection and dissociation phases of 5 and 10 min, respectively. In order to ensure full reconstitution of the surface after regeneration, the equilibration phase was increased to 30 min. Three buffer blanks were included in each analysis and their average signals were subtracted from the processed sample responses (double referencing). Data processing and kinetic analysis was performed using Scrubber (version 2.0a, BioLogic Software Pty Ltd) and CLAMP (version XP)⁴¹. Each data set was globally fitted to either a Langmuir 1:1 interaction model (C3(H₂O), C3b, and C3dg) or a surface heterogeneity model (C3 native) to obtain the association and dissociation rate constants (kₒₙ and k_{off}, respectively). The equilibrium dissociation constant (K_{d}) was calculated from the kinetic parameters as K_{d}=k_{off}/kₒₙ.

In order to prevent native C3 from being hydrolyzed, freshly purified plasma C3 was selectively precipitated by dialysis against MES pH 6.0 and stored at −80° C. Immediately before analysis, the precipitate was reconstituted in running buffer and kept on ice until injection. To verify that the observed SPR signal for native C3 was not generated by contamination with C3(H₂O), Mono-S cation exchange chromatography was used to further purify the experimental protein sample. The content of residual C3(H₂O) was quantified by an ELISA method using the hydrolyzed C3-specific monoclonal antibody C3-9⁴⁰, and was found to be less than 2% of total protein weight. The purified native C3 fraction was desalted into running buffer and injected onto an Efb-C sensor chip, which resulted in a clear binding signal within the expected experimental range.

SPR was also used to test for the induction of conformational change in C3b upon binding to Efb-C. In this experiment, 7,000 RU of monoclonal antibody C3-9 were immobilized as described above. Analyte solutions consisting of 50 nM Efb-C, C3b, or a stoichiometric Efb-C/C3b complex were prepared in PBS-T and injected for a total of 2 min followed by a 2 min dissociation phase and regeneration with 10 mM glycine pH 2.0 for 60 s.

### Results:

### The Crystal Structure of Efb-C Reveals a Novel Complement Regulatory Motif

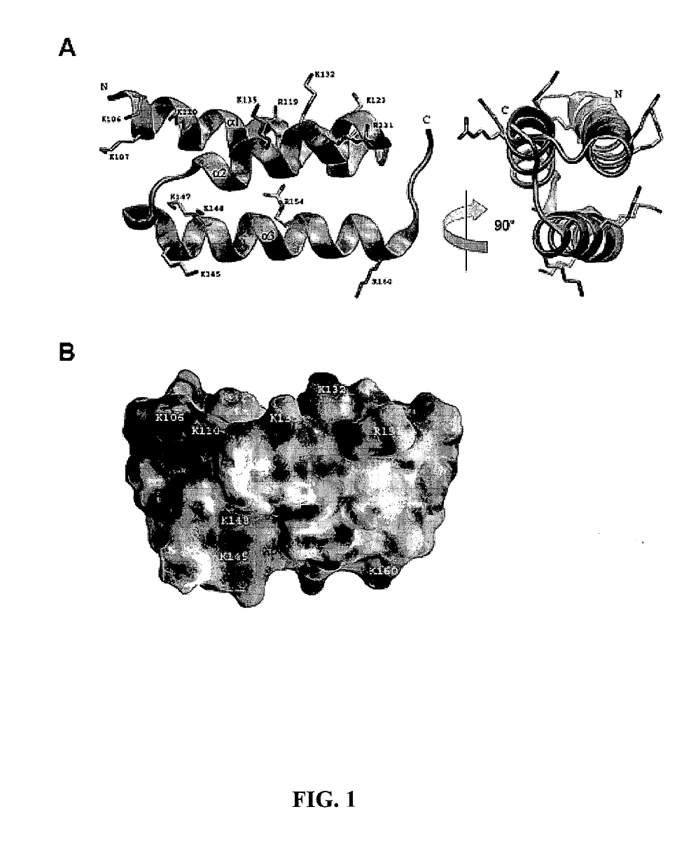

To gain structural insight into the molecular recognition of RCA proteins and their role in complement cascade inhibition by S. aureus, the crystal structure of the C3-binding C-terminal region of Efb (Efb-C) was determined. The structure of Efb-C was determined using multiwavelength anomalous diffraction (MAD) data collected from crystals of selenomethionyl-substituted protein and refined to 1.25 Å limiting resolution with R_{cryst} and R_{free} values of 21.1 and 21.8%, respectively (Table 1 and Methods). The overall dimensions of Efb-C are approximately 40×25×20 Å (FIG. 1A), where the N-terminal al helix (K106 to H125) is connected trough a short loop with the α2 helix (V127 to L139), which is then followed by C-terminal α3 helix (K145 to Q161) that terminates in a random coil conformation (G162 to R165). The packing of the all three helices in a coiled coil is canonical, with most of the non-polar side chains directed inward; however, there is an obvious preponderance of solvent-exposed basic residues in Efb-C (FIG. 1B).

TABLE 1
Data Collection Statistics, Determination, and Refinement
of the Efb-C and Efb-C/C3d Crystal Structures
Data Collection^{a}

| | Efb-C/C3d |
|---|---|
| Space group | P4₁ |
| Unit cell dimensions (Å) | a = b = 90.9, c = 122.2 |
| Molecule/ASU | 2 |
| Resolution limits (Å) | 50-2.2 |
| Completeness (%) | 98.8 (83.4) |
| Total reflections | 244,752 |
| Unique reflections | 49,718 |
| R_{merge} (%)^{b} | 9.2 (47.3) |
| <<I>/<σI>> | 12.7 (1.8) |

Refinement Statistics

| | Efb-C | Efb-C/C3d |
|---|---|---|
| R_{cryst}/R_{free} (%)^{c} | 21.1/21.8 | 18.1/23.1 |
| RMSD from ideality | | |
| Bond length (Å) | 0.006 | 0.024 |
| Bond angle (°) | 0.93 | 1.87 |
| Dihedral angle (°) | 18.7 | 18.5 |
| Ramachandran core (disallowed) (%) | 96.5 (0) | 93.4 (0) |
| Average B factor (Å²) | 19.0 | 43.5 |
| RMSD of B factor (Å²) | 1.2 | 11.4 |
| Proteins atoms modeled | 987 | 5674 |
| Ordered solvent molecules | 155 | 317 |

^{a}Data collection and X-ray diffraction analysis of Efb-C has been published separately²⁷
^{b}R_{merge} = ΣₕΣᵢ|Iᵢ(h) − <I(h)>|/ΣₕΣᵢIᵢ(h), where Iᵢ(h) is the i^{th} measurement of reflection h and <I(h)> is a weighted mean of all measurements of h.
^{c}R = Σₕ|F_{obs}(h) − F_{calc}(h)|/Σₕ|F_{obs}|. R_{cryst} and R_{free} were calculated from the working and test reflection sets, respectively.
The test set constituted 5% of the total reflections not used in refinement.

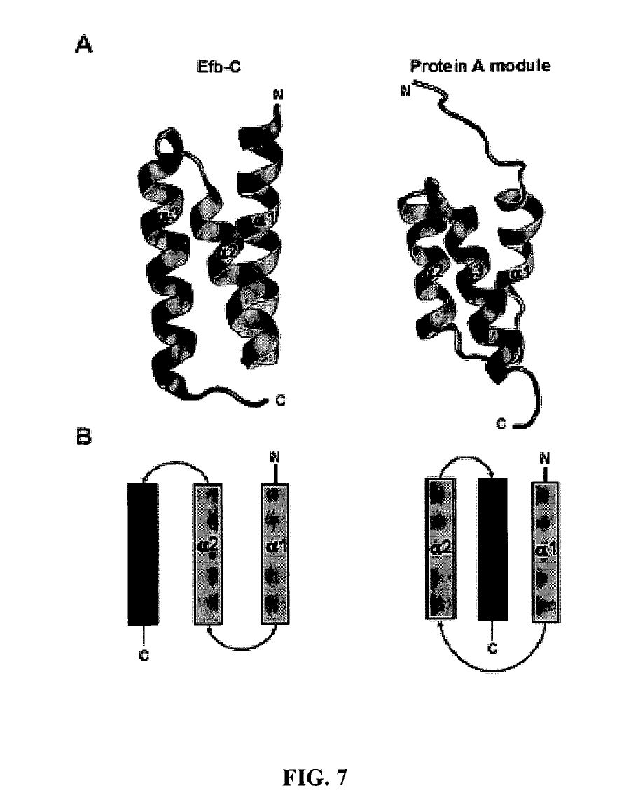

All currently known examples of complement regulatory proteins are comprised of the SCR (short consensus repeat/complement control protein) beta-type fold', including 1H, its viral homologue vaccinia virus complement control protein (VCP), MCP/CD46, DAF/CD55, CR1/CD35, and C4BP. In sharp contrast, the Efb-C structure is entirely helical and therefore defines a completely novel fold-class for complement regulatory proteins. This type of all-helical structure is not unprecedented for extracellular Staphylococcal proteins, however, as the Efb-C structure is reminiscent of S. aureus protein A modules (FIG. 7). Nevertheless, the topological arrangement of the helices in Efb-C is distinct from that found in protein A so that an evolutionary relationship between these two classes of virulence factors is unlikely.

Insights into Complement Recognition Provided by the Efb-C/C3d Crystal Structure

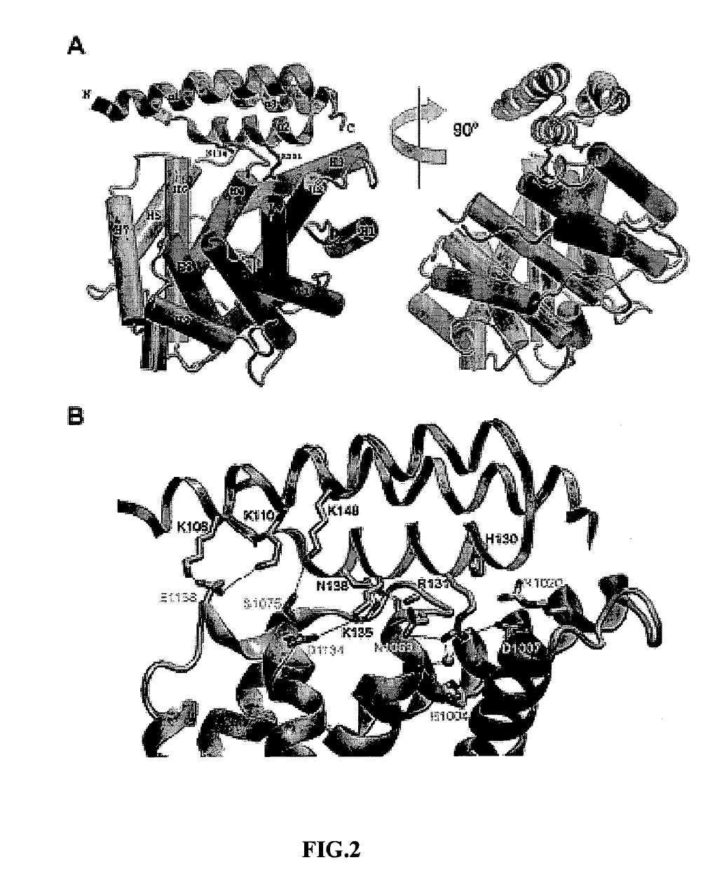

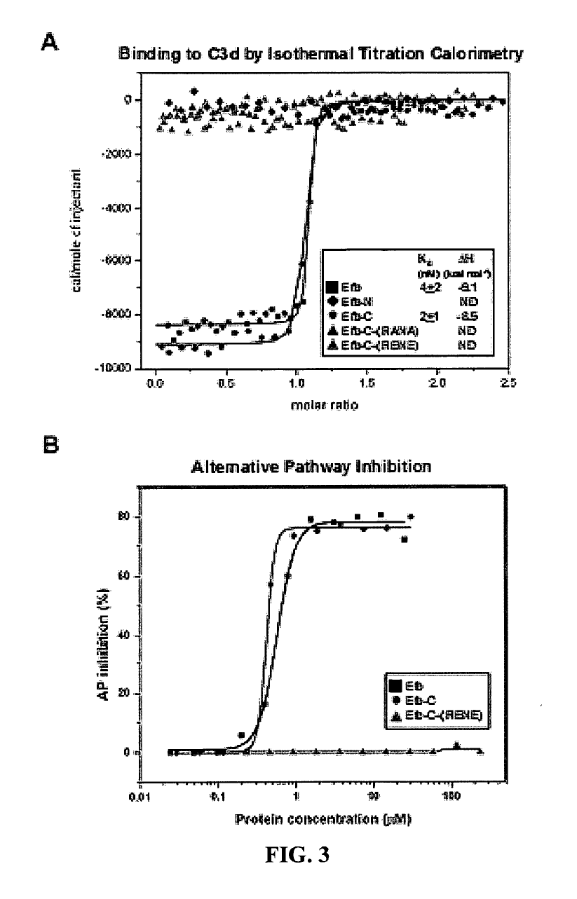

The crystal structure of Efb-C bound to recombinant C3d was determined. The Efb-C-C3d structure was refined to 2.2 Å limiting resolution with R_{cryst} and R_{free} values of 18.1 and 23.1%, respectively (FIG. 2a and Table 1). A single Efb-C molecule bound to C3d in the complex, and comparison of Efb-C in both its free and bound state revealed minimal structural changes. In particular, ordered character was observed until residue T101 of Efb-C when bound to C3d, a fact that most likely reflects stabilization of the N-terminal al helix in Efb-C through interaction with C3d. Overall, 61 of 65 residues aligned within 2.5 Å and an r.m.s.d. of 0.47 Å when comparing the free and bound states of Efb-C. This suggested that the C3d binding site on Efb-C is preformed.

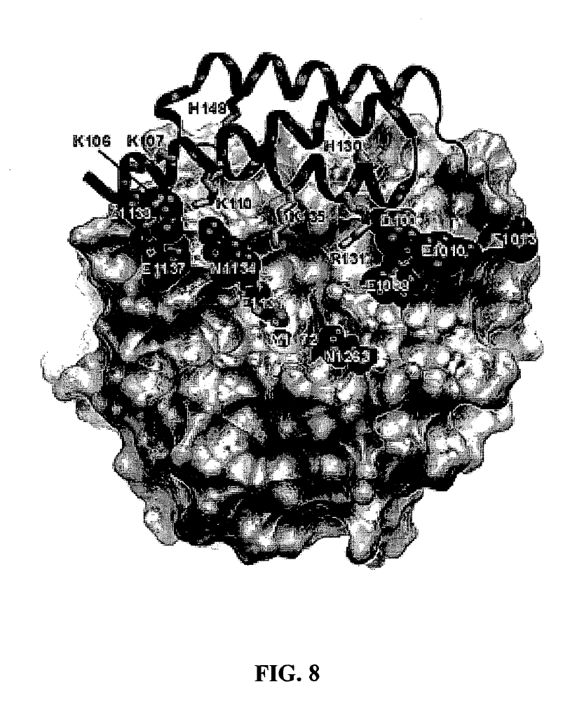

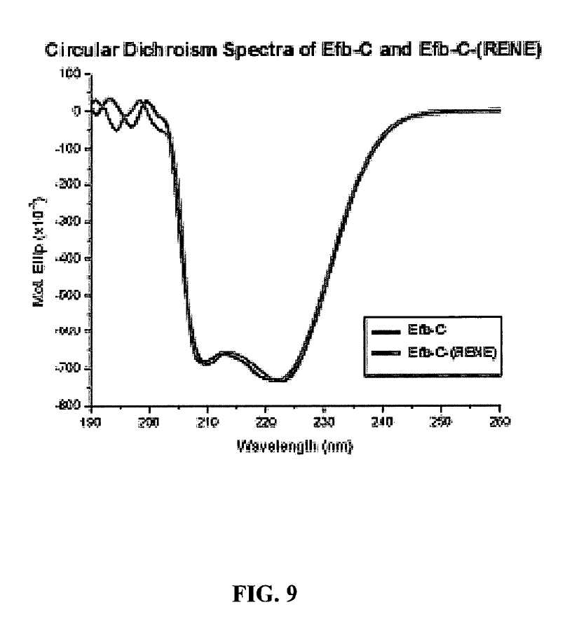

In the co-crystal, C3d adopted its canonical dome-shaped helical structure¹⁷ and a single Efb-C molecule was found at the periphery of the conserved acidic pocket comprised of residues D1029, E1030, E1032, D1156, E1159, and E1160 on the concave surface of C3d (FIG. 2b and FIG. 8). A majority of the contacting residues were donated from Efb-C helix α2 (H130, R131, K135, and N138), although sidechains from helices al (K106 and K110) and α3 (K148) also appeared capable of serving a minor role in forming the Efb-C-C3d complex. In contrast to the interacting residues in Efb-C, the C3d residues found at the complex interface were separate in primary sequence but came together in the folded protein. In particular, residues from the loops connecting the H2-H3, H4-H5 and H6-H7 helices formed specific contacts with Efb-C. The Efb-C-C3d interface demonstrated excellent shape complementarity with a relatively large buried surface area of 1967 Å² that accounted for 44% of the available Efb-C surface. This surface compatibility was particularly striking in the case of Efb-C R131, which was tucked deeply within a solvent-lined pocket of C3d comprised of residues H1026, D1029, N1091, and L1092 (FIG. 2b). Closer inspection of the intermolecular hydrogen bonds also revealed an intricate network between N138 of Efb-C and the main chain atoms of residues V1090, I1093, and I1095 that formed the H4-H5 loop of C3d (FIG. 2b).

### Efb-C Inhibits Complement Activation by Altering the Solution Conformation of Native C3

Previous studies provided evidence that Efb-C was both necessary and sufficient to block complement pathway activation^{5,6}, and the crystal structures presented here suggested that this inhibition is derived from the ability of Efb-C to form a specific, high-affinity complex with the C3d domain of C3. Site-directed mutagenesis studies confirmed the importance of both R131 and N138 in forming the Efb-C/C3d complex (FIG. 3A) and allowed the generation of Efb-C mutants incapable of binding to C3d. To understand the role of the Efb/C3d complex in complement inhibition more thoroughly, the activities of Efb, Efb-C, and the non-functional R131E_N138E double mutant of Efb-C (denoted Efb-C-(RENE)) were measured in a series of quantitative, functional assays that monitor the generation and surface deposition of C3b through either the classical (antibody-dependent) or alternative (antibody-independent) pathways (FIG. 3B). These experiments revealed that the inhibitory properties of Efb are directly related to C3d binding, since Efb and Efb-C had essentially identical activities while the double mutant had no activity in either assay. Furthermore, while Efb required a 50-fold molar excess over C3 to achieve an IC50 concentration for the classical pathway, both Efb (IC50 ˜560 nM) and Efb-C (IC50 ˜410 nM) were essentially equimolar with C3 in the alternative pathway assay. This indicated that the principal effect of Efb is through blocking activation of the alternative pathway, and that the observed effect on the classical pathway most likely reflected Efb-mediated inhibition of the alternative pathway self-amplification loop, which contributes a majority of the classical pathway generated C3b.

### Further Results and Discussion of FIG. 3A: Thermodynamic Analysis of the Efb/C3d Interface

Isothermal titration calorimetry was used to monitor the binding of full-length Efb, Efb-N, and Efb-C as well as its mutants to C3d (FIG. 3A, values inset). The observed equilibrium dissociation constant (K_{d}) for both Efb and Efb-C binding to C3d showed high affinity, 1:1 binding in the nanomolar range (K_{d}=2±1 nM), and similar values enthalpic parameters (ΔH) of −9.0 and −8.5 kcal mol⁻¹ were observed for full-length Efb and Efb-C, respectively. These results are in agreement with previous studies and demonstrate that Efb-C is necessary and sufficient for binding to C3d⁶ and that Efb-N contains only the fibrinogen-binding activity of Efb, since this truncated protein had no measurable affinity for C3d in this assay⁸. Two site-directed mutants of Efb-C were constructed, wherein both R131 and N138 were substituted with either alanine or glutamic acid, respectively, and the C3d-binding properties of the resulting proteins were examined using the same assay (FIG. 3A, values inset). In both cases, the results of these studies supported the Efb-C/C3d interactions as determined from the crystal structure, since neither double mutant had detectable binding to C3d despite the fact that each was structurally in-tact (Data Not Shown and Supplementary FIG. 3). No binding to C3d was detected for the form of Efb-C where both R131 and N138 were mutated to alanine. This highlights the importance of the specific non-covalent interactions formed by these sidechains (FIG. 2B) and suggests that the contacts formed between the other residues at the Efb-C/C3d interface are mostly ancillary in nature, as these themselves were insufficient to drive complex formation.

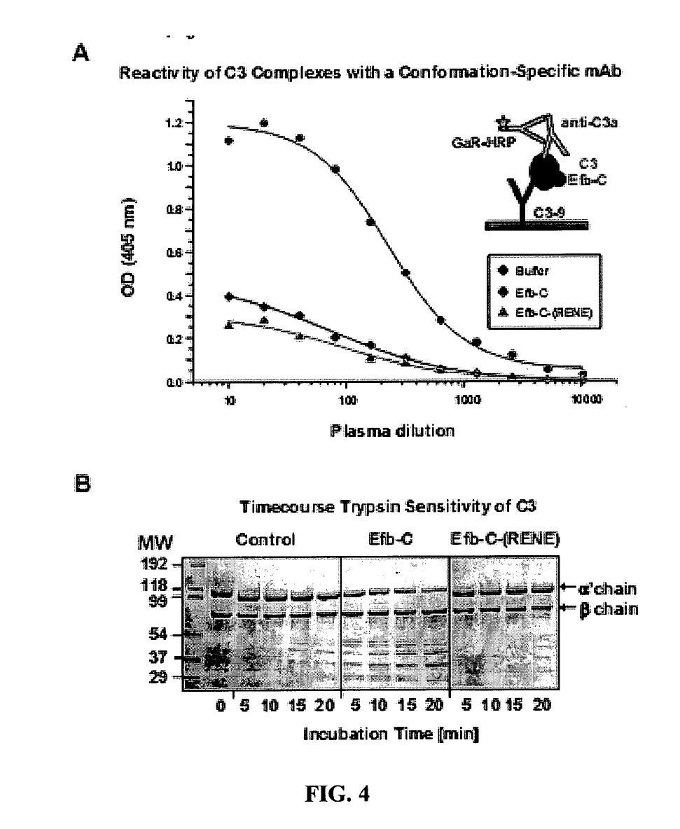

To define further the functional basis for Efb-mediated inhibition of the alternative pathway, the C3 contained in human plasma was incubated with either Efb-C, Efb-C—(RENE), or buffer alone, and the reactivity of each sample over a 1,000-fold dilution range was examined with a well-established panel of monoclonal antibodies that are sensitive to both the composition and conformation of C3 and/or the various intermediates of its activation and degradation pathways. Treatment of plasma with Efb-C resulted in a marked increase in the reactivity of bound C3 with the monoclonal antibody C3-9, which detects a neoantigen that becomes exposed during C3 activation to hydrolyzed C3 (C3(H₂O)) and C3b. The fact that C3 captured in this manner was recognized by both the anti-C3a (FIG. 4A) and anti-C3b antibodies throughout the entire dilution series indicated that C3 bound to Efb-C was not being processed into C3b, but instead suggested that Efb-C binding altered the conformation of C3 and led to an increased exposure of the C3-9 epitope.

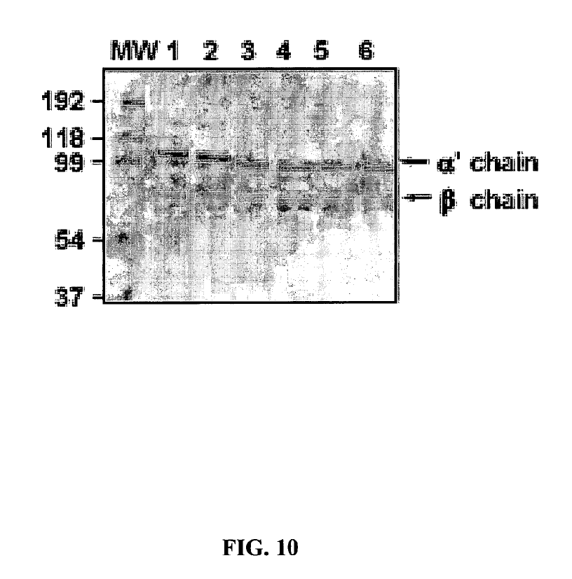

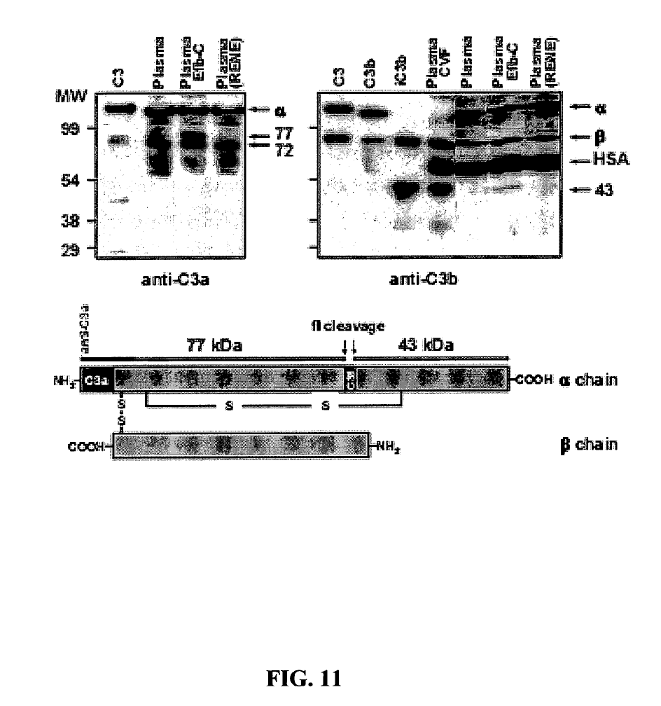

Protease sensitivity assays have long been used as a biochemical probe to assess the folding state and structure of proteins. Limiting proteolysis by trypsin revealed that C3 bound to Efb-C was rapidly processed into a series of lower molecular-weight fragments whereas only small amounts were observed in the Efb-C-(RENE) and control samples, even after a prolonged exposure to trypsin (FIG. 4B and FIG. 10) A similar effect could be observed in plasma samples where complement activation was blocked by the addition of EDTA. Here, addition of Efb-C resulted in sensitivity of C3 to proteolysis that was not detected in either the Efb-C-(RENE) or untreated controls (FIG. 11). Together, these results indicate that Efb-C binding results in a conformation of C3 that exposes an epitope found in C3(H₂O), but is unable to participate in downstream complement activation.

### Efb-C Displays a Unique Binding Preference for Native Forms of C3

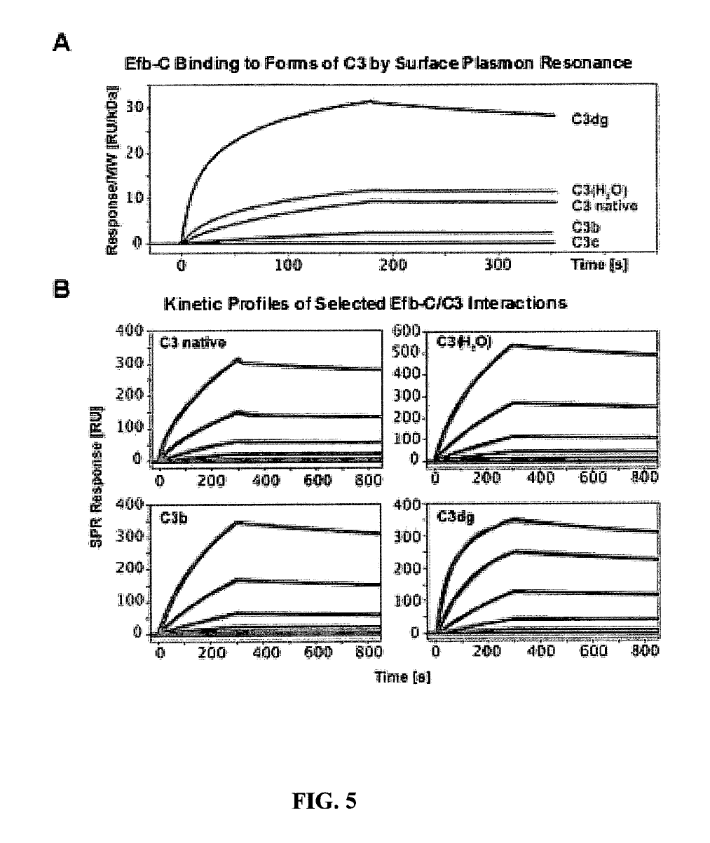

The recent crystal structures of C3, C3b, and C3c^{18,19}, along with considerable biochemical data have demonstrated that activation of C3 to C3b is accompanied by substantial conformational changes^{1,20-22}. These conversions lead to an exposure of previously hidden binding sites and to changes in affinity for an array of receptors and regulatory proteins in C3b relative to C3, which has few physiological ligands in its native state. In light of this knowledge, the Efb-C-induced conformational changes in C3 identified above suggested that Efb-C may also display differential binding to C3 and its various intermediates that represent different states of its activation pathway. To test this, Efb-C was immobilized on a surface plasmon resonance biosensor and a series of binding assays was conducted to determine the relative affinities of Efb-C for C3, hydrolyzed C3 (C3(H₂O)), C3b, C3c, and C3dg, all of which are known to have distinct conformations and/or ligand-binding properties (FIG. 5A). Consistent with the isothermal titration calorimetry shown in FIG. 3, Efb-C was observed to bind to all forms of C3 that included the thioester-containing C3d domain; oppositely, no binding to the C3c fragment was detected.

To expand upon these observations, full kinetic profiles for those C3 fragments capable of binding to Efb-C were determined (FIG. 5B and Table 2). All C3 fragments featured affinity constants (KO in the low nanomolar range and, furthermore, the data were internally consistent with the solution calorimetry results shown in FIG. 3A. Nevertheless, the association (kₒₙ) and dissociation rate constants (k_{off}) for each fragment varied significantly. To begin, C3dg featured a substantially higher association rate constant than all other C3 analytes, which might be attributed to the unimpeded accessibility of the Efb-C binding site in this comparatively small fragment. Second, even though the dissociation constants of both native C3 and C3(H₂O) are similar, their kinetic profiles are quite distinct. C3(H₂O) bound Efb-C more quickly, but this complex also dissociated at a rate over 70% greater than that of native C3. Finally, despite the fact that C3(H₂O) has C3b-like functional properties ²⁷, the affinity of Efb-C for C3b was found to be much lower than expected. Here, an approximately three-fold decrease in affinity of C3b compared to native C3 and C3(H₂O), and a nearly ten-fold difference to C3dg was observed. Substantial differences in both the association and dissociation rates contributed to the lower affinity. Such a binding preference is believed to be quite unique, since all known complement regulatory proteins bind preferentially, if not exclusively, to C3b².

TABLE 2
Surface Plasmon Resonance Kinetic Profiles
of Selected C3 Fragments Binding to Efb-C^{a}

| Target | Analyte | kₒₙ (10⁴ M⁻¹s⁻¹) | k_{off} (10⁻⁴ s⁻¹) | K_{d} (nM) |
|---|---|---|---|---|
| Efb-C | C3 native^{b} | 3.60 ± 0.03 | 0.99 ± 0.19 | 2.8 ± 0.5 |
| | C3 (H₂O) | 8.89 ± 0.87 | 1.69 ± 0.14 | 1.9 ± 0.0 |
| | C3b | 2.39 ± 0.13 | 2.09 ± 0.01 | 8.8 ± 0.5 |
| | C3dg | 28.44 ± 11.89 | 2.18 ± 0.42 | 0.8 ± 0.1 |

^{a}All analytes were injected over immobilized Efb-C as threefold linear dilution series (200-0.9 nM for C3b, 67-0.09 nM for other analytes). Data were fit to either a 1:1 Langmuir (C3(H₂O), C3b, and C3dg) or a surface heterogeneity binding model (C3 native).
^{b}Kinetic rate constants of the primary binding site (~90% of the total binding).

Further Results and Discussion of FIG. 5 and Table 2: SPR Characterization of Efb-C Interactions with C3 Proteins.

The sensorgrams of C3(H₂O), C3b, and C3d binding to Efb-C were well described by a 1:1 Langmuir binding model, and indicative of a single interaction site for these proteins on Efb-C. Only native C3 showed a small but significant deviation from the Langmuir term, and consequently this sensorgram was analyzed by a surface heterogeneity model. Typically, deviations from an ideal 1:1 curve fit might be derived by immobilization-induced heterogeneities. Since all other C3 fragments followed a 1:1 binding model, heterogeneity of the immobilized Efb-C is unlikely as source for the observed deviations of native C3 in this case. Instead, a secondary binding site, or conformational adaptations during the binding process are more reasonable explanations. Even though the strong affinity constants required rather harsh regeneration conditions (0.1% SDS), the immobilized Efb-C was found to refold quickly with no significant loss of activity. As a consequence, repetitive sample injections showed a high reproducibility. Since the analysis was carried out at low protein densities and at high flow rates, mass transport limitation was ruled out as a cause for the small deviations. Indeed, inclusion of a mass transport term in the kinetic model did not improve the fit, and higher flow rates did not change the kinetic profile significantly.

### A Potential Structural Basis for Differential Recognition of C3 Intermediates by Etb-C

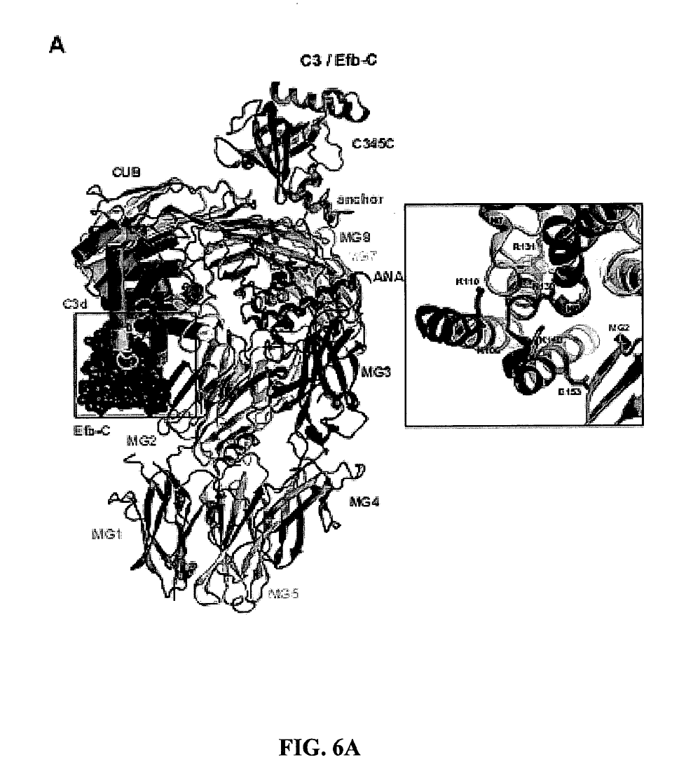

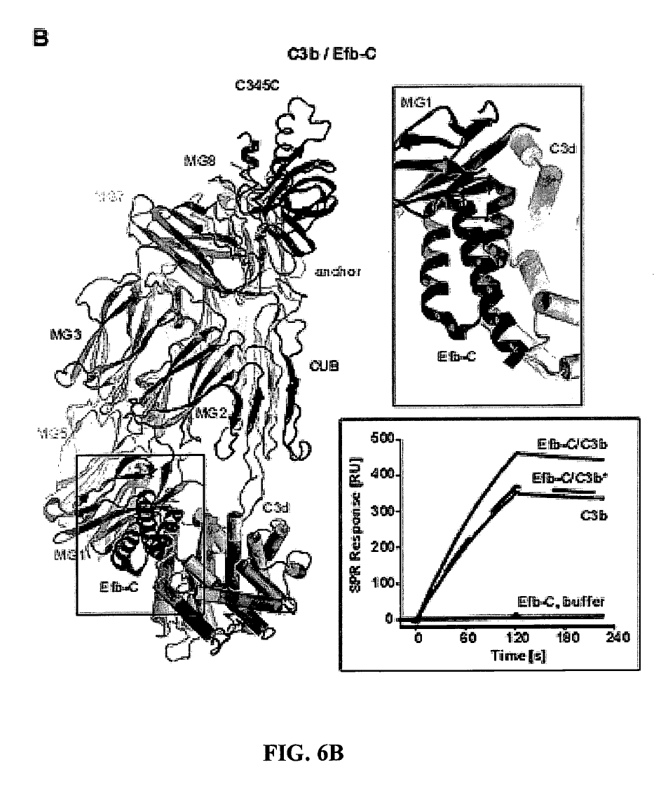

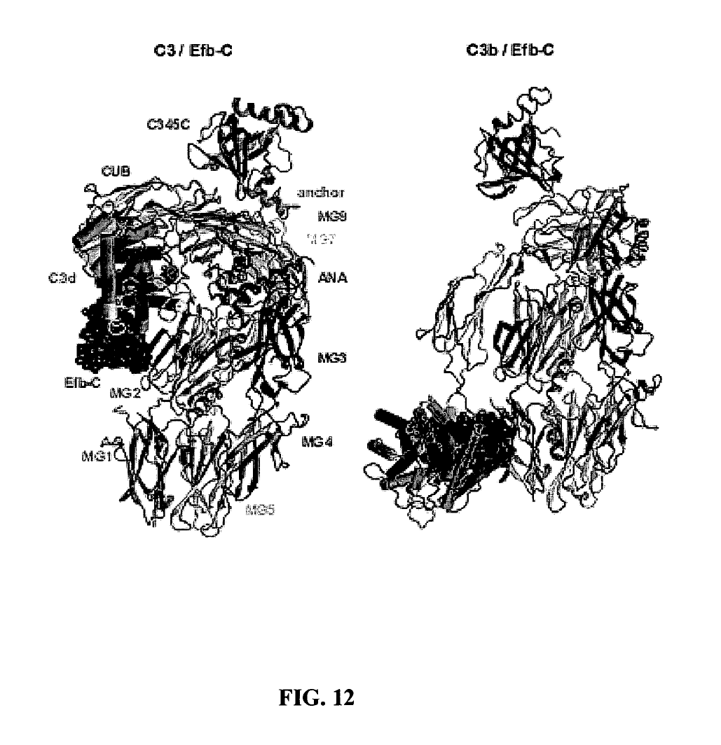

Several observations that provide a structural basis for the binding preferences and conformational effects of Efb-C on C3 can be found when the Efb-C/C3d complex presented in FIG. 2 is interpreted within the context of the full-length C3 structure (FIG. 6A). First, the Efb-C binding site on C3d includes the H4-H5 and H6-H7 loop regions that adopt altered conformational states during the activation of C3 to C3(H₂O) and C3b, as measured by hydrogen/deuterium-exchange mass spectrometry methods. Notably, N138 of Efb-C, which is critical to the Efb-C/C3d interaction (FIG. 2), makes extensive contacts with the H4-H5 loop of the C3d domain. The thermodynamic and kinetic stability of the Efb-C/C3 complex, as determined by both calorimetry and plasmon resonance studies (FIGS. 3 and 5), suggests that Efb-C binding to C3 may lock these important loops in a subsequently inactive state. Second, in addition to its primary contacts with the C3d domain as described above, Efb-C appears poised to make additional interactions with the surface of C3 (FIG. 6A, Inset). In particular, residues V146, M149, V 150, and E153 from Efb-C helix a3 are all less than 4 Å distance from sidechains donated by the second α2 macroglobulin domain of the C3 β-chain. The existence of these additional interactions may well contribute to the greater affinity of Efb-C for C3 and C3(H₂O) when compared to C3b, since the recent crystal structures of C3b reveal that the cognate C3d domain undergoes a 65.7 Å translation and a rotation of 103.6° compared to its location in C3. This large conformational change that accompanies C3 activation makes any additional contacts between Efb-C and the MG2 domain of C3 physically unlikely (FIG. 6B, FIG. 12).

An equally striking observation is made when the same approach is used to analyze the Efb-C binding site in C3b (FIG. 6B). Here, Efb-C appears to undergo a tremendous steric clash with the first a 1 macroglobulin domain (MG1) of the C3b β-chain despite the fact that the C3d domain is relatively more exposed in this structure than in native C3 (FIG. 6B, Inset Top). It is difficult to imagine how such a structure could exist, yet the SPR binding studies above demonstrate that Efb-C still forms a potent complex with C3b (FIG. 5 and Table 2). Without being limited by any explanation of mechanism, it may be that Efb-C may also induce a structural change in C3b. To test for this possibility, an SPR binding assay was performed using the immobilized monoclonal antibody C3-9, and examined its reactivity toward C3b and a preformed Efb-C/C3b complex (FIG. 6B, Inset Bottom). The observed signal intensity for the Efb-C/C3b complex was approximately 30% greater than the anticipated signal calculated from the ideal mass increase of the complex (˜5%), even though C3-9 does not recognize Efb-C. The increased reactivity of the Efb-C/C3b complex relative to C3b alone indicates that the C3-9 epitope becomes more exposed in the complex, and is consistent with the induction of an additional conformational change in C3b when bound to Efb-C. While the primary mechanism of Efb inhibition seems to be centered on blocking the generation of active C3b, the abundance of C3 in the human plasma argues that any single mechanism of inhibition is unlikely to be totally effective. Therefore, this additional effect on C3b may contribute physiologically to immune evasion by S. aureus.

### Conclusions

The most common complement inhibitory strategy employed by various pathogens involves the indirect acquisition of host RCA proteins, where bacterial binding proteins recruit soluble 1H, factor like H protein-1 (FHL-1) or C4BP to the bacterial cell surface²⁴. These surface complexes not only retain the complement inhibitory activities of the host RCA molecules and provide an effective measure of immune evasion, but also appear to result in a physical barrier that efficiently separates the bacterial cell surface from the site of the attack. Staphylococcus aureus appears to have evolved an alternative strategy where secreted proteins are used directly to block the various steps needed in the initial activation of the complement response. Indeed, the recent identification of a Secreted Complement Inhibitory protein (SCIN⁷) that appears to function in a manner distinct from Efb indicates that S. aureus has evolved multiple, mechanistically-distinct proteins capable of inhibiting the complement system.

The results presented in this example provide evidence for a novel mode of complement inhibition. In this mechanism, Efb-C blocks the formation of the functional C3b opsonin by binding tightly to the thioester-containing domain of native C3, and by perturbing the overall solution conformation of the molecule to one that is incapable of being processed into C3b. Moreover, despite the fact that Efb-C binds preferentially to native forms of C3, Efb-C appears to recognize C3b with high affinity and to induce a conformational change in this activated complement component as well.

### Example 2

### Identification of a Novel Secreted Protein from S. aureus. That Binds C3 and Inhibits Complement Pathway Activation

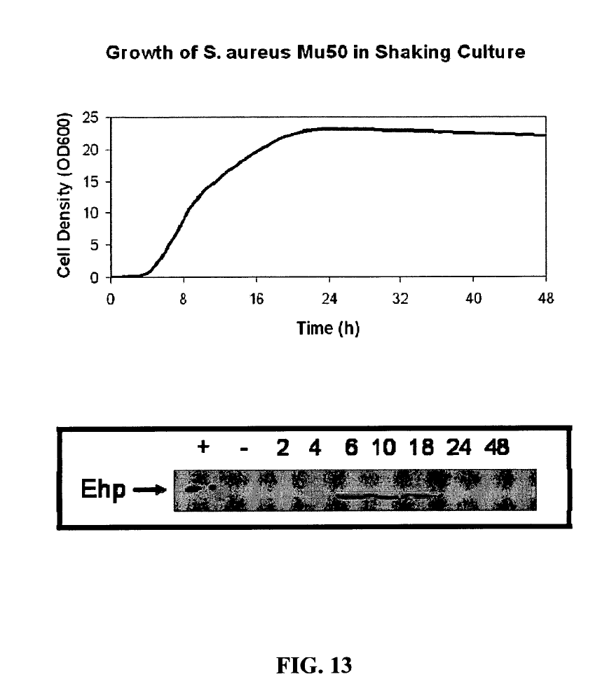

The uncharacterized protein SAV 1155 (GenBank Accession Number NP_{—}371679 and denoted Ehp for Extracellular Fibrinogen-binding Protein Homologous Protein in the remainder of the text) was identified during a genome-wide scan to identify potential Type-I Signal Peptidase-dependent secreted proteins from S. aureus. The Ehp open-reading frame encodes a 109 residue polypeptide, although the primary sequence of this protein contains a high-probability Type-I signal peptidase cleavage site between residues 29 and 30 and is predicted to yield an 80 residue mature protein with a deduced molecular weight of 9.558 kDa. To determine whether Ehp was in fact secreted from S. aureus, polyclonal antisera were raised against a recombinant form of Ehp corresponding to the predicted mature protein. The immune sera were used to test for the presence of Ehp in the growth medium of S. aureus cultures at various time points (FIG. 13). It was found that Ehp was efficiently secreted into the medium, and was present at highest levels during log-phase growth in non-selective medium (6-18 hrs). These results demonstrated that Ehp is secreted from actively growing S. aureus. Furthermore, this particular expression profile is a hallmark of quorum sensing-regulated virulence factors from S. aureus and suggests that Ehp might play an important role in Staphylococcal pathogenesis.

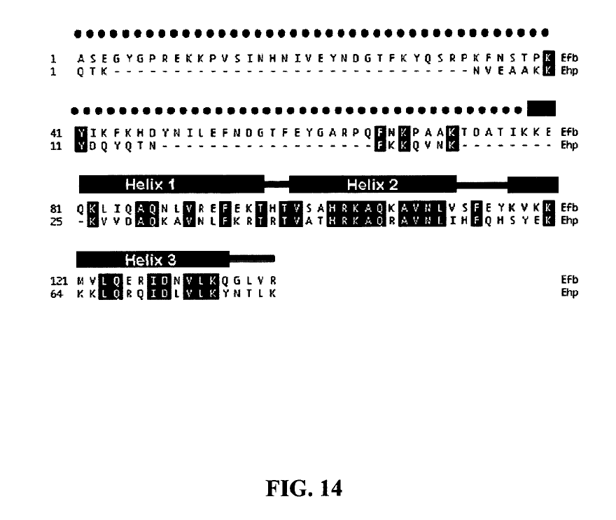

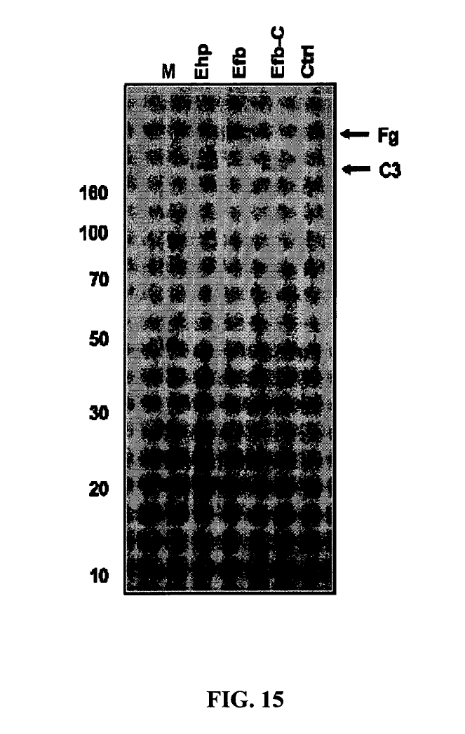

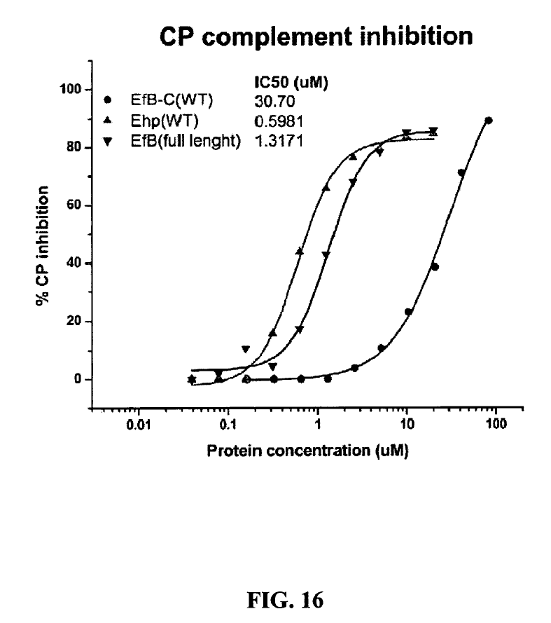

Because no functional data were available for Ehp, the relationship of this protein to previously studied molecules was examined based upon sequence similarities. BLAST searches of the non-redundant (NR) protein database revealed that Ehp was homologous to the 16 kDa C3-binding, extracellular fibrinogen-binding protein, Efb, from S. aureus. Clustal sequence alignment between Ehp and Efb revealed that the region of homology between these two proteins was confined to the carboxyl-terminal half of Efb (FIG. 14), which represents the Efb-C region whose crystal structure was presented above in FIG. 2; in fact, Ehp is 44% identical to the 61 residues comprising Efb-C, suggesting that it has a very similar fold to Efb-C. Since Efb-C was recently shown to possess potent C3-binding and complement inhibitory activities in vitro, these observations indicated that Ehp is a C3-binding complement inhibitory protein from S. aureus. To test this hypothesis directly, Ehp, Efb, and Efb-C were coupled to sepharose resin and the derivatized resins were incubated with a 1:100 dilution of human serum in a physiological buffer. Following several brief washes to remove loosely bound contaminants, the specifically-bound serum proteins were separated under non-reducing conditions and analyzed by Coomassie-stained SDS-PAGE and LC-MS/MS techniques (FIG. 15). Ehp, Efb, and Efb-C each bound a protein of roughly 180 kDa that was subsequently identified by LC-MS/MS as C3; likewise full-length Efb, which contains an N-terminal Fg-binding domain, bound specifically to a slower migrating Fg species (LC-MS/MS) that was not recognized by either Efb-C or Ehp. The fact that Ehp could bind specifically to C3 suggested that Ehp may also function as an inhibitor of complement pathway activation, similar to Efb. As shown in FIG. 16, Ehp exhibited potent inhibition of classical activation of the complement pathway with an IC50 of 0.59 μM, as determined by an ELISA-based assay detecting fixed C3b to Ag-Ab complex. This level of inhibition is approximately twice as effective on a molar basis as Efb, and nearly 50 times that seen for Efb-C. A similar level of inhibition was also observed for the alternative pathway of complement activation, but is not presented here in the interest of brevity. Together, these results indicate that Ehp is a potent inhibitor of complement activation pathways and along with Efb defines a novel family of secreted complement regulators.

### Example 3

### Quantitative Analyses of C3 Interactions with S. aureus Proteins Using Isothermal Titration Calorimetry and Recombinant C3d

Conversion of C3 to C3(H₂O) and C3b is central to activation of the downstream complement response. Functional mapping of regions that participate in structural rearrangements in C3 during its activation has revealed that most of the regions that undergo conformational change are loops localized around the C3d domain, a result that is consistent with previous observations that C3d is critical to both the activation and regulation of the complement response. Appropriately, Efb binds to human C3, potently blocks C3 activation through both the classical and alternative pathways, and this interaction is localized to the C3d region. Previous studies by others also suggested an equilibrium dissociation constant (K_{d}) of approximately 240 nM for the Efb/C3d interaction, as determined by fluorescence quenching of tryptophan residues. To quantitatively assess the interactions between Efb (and Ehp) and C3d from an independent approach and with minimal assumptions of the mechanisms involved, a sensitive, isothermal titration calorimetry assay (ITC) was developed. The enthalpy change following titration of purified samples of Efb, Efb-C, and Ehp into a fixed quantity of purified, recombinant C3d expressed in and purified from E. coli was measured, and the resulting data were fit to binding isotherms to determine the stoichiometry (N) and equilibrium dissociation constant (K_{d}) for each interaction.

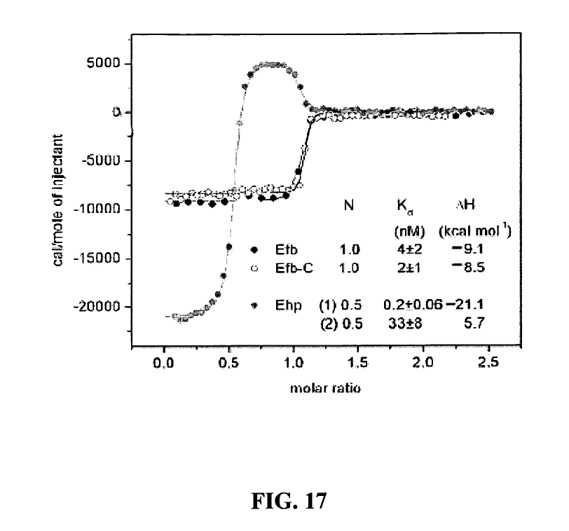

Based on the results shown in FIG. 17, several important conclusions regarding C3 binding by S. aureus proteins have been drawn. First, the results demonstrate that Efb-C is both necessary and sufficient for binding to C3d. Efb-N, which is responsible to the Fg-binding activity of this protein had no measurable affinity for C3d in this assay, and this is consistent with results published previously by other investigators. Second, the equilibrium dissociation constant (K_{d}) for both Efb and Efb-C binding to C3d in this assay was determined at roughly 2 nM; this value is approximately 120-fold tighter than the figure previously reported (Lee, L. Y. L., Liang, X., Hook, M., and Brown, E. L. 2004. Identification and characterization of the C3 binding domain of the Staphylococcus aureus extracellular fibrinogen-binding protein (Efb). J Biol Chem 279:50710-50716). This difference likely reflects the fact that ITC is an inherently more accurate assay than fluorescence quenching and suggests that the previous experiments were done at concentrations of Efb-C far above the actual K_{d} for the interaction. Finally, and consistent with the observations presented above in FIG. 15, it was observed that Ehp also forms a potent complex with C3d. Together, these results demonstrate that the helical-bundle domain present in the secreted S. aureus proteins Efb and Ehp is responsible for forming a nanomolar-affinity complex with C3d that blocks downstream complement function.

During the course of analyzing the ITC data, it was observed that the interaction between Ehp and C3d was not entirely well described by an A+B [symbol] AB equilibrium, as is the case for Efb/C3d. This prompted a reevaluation of the Ehp/C3d binding data through an alternative model where Ehp contains two non-equivalent C3d binding sites. Fitting with this revised formalism explains the experimental ITC data quite well, and suggests K_{d} values of approximately 200 pM for the first and 33 nM for the second C3d-binding site in Ehp, respectively. Since this titration saturates at approximately 1:1 molar ratios of Ehp and C3d, these data support a model where Ehp and C3d interact physically at two distinct sites. In fact, closer examination of the data revealed that an approximately 3 kcal/mol change in AG between the first and second sites is accompanied by a large, 27 kcal/mol unfavorable change in enthalpy release; this result is consistent with a large entropic gain in the second site association, and suggests that either a substantial release in ordered solvent from the second Ehp/C3d interface, or a conformational change, or some combination of these two factors accompanies this interaction. Taken together, these data indicate that Ehp is not simply a more compact version of Efb and suggest that there are likely to be several important structural and biochemical differences between the Ehp/C3 and Efb/C3 complexes. Furthermore, it is believed that these data provide the first functional explanation as to how Ehp is at least as potent as Efb at inhibiting complement activation, even though Ehp lacks the N-terminal Fg-binding domain that contributes increased potency to Efb when compared to Efb-C alone (FIG. 16).

### Example 4

### Bacterial Production of Peptides

The following peptides were produced by bacterial expression, as described below.

(1) Efb: S. aureus strain Mu50, GenBank
Accession Number P68799 (residues 28-165):
(SEQ ID NO: 3)
ASEGYGPREKKPVSINHNIVEYNDGTFKYQSRPKFNSTPKYIKFKHDYNI
LEFNDGTFEYGARPQFNKPAAKTDATIKKEQKLIQAQNLVREFEKTHTVS
AHRKAQKAVNLVSFEYKVKKMVLQERIDNVLKQGLVR;
(2) Efb-C: S. aureus strain Mu50, GenBank
Accession Number P68799 (residues 93-165):
(SEQ ID NO: 4)
FNKPAAKTDATIKKEQKLIQAQNLVREFEKTHTVSAHRKAQKAVNLVSFE
YKVKKMVLQERIDNVLKQGLVR;
(3) SAV1155/Ehp: S. aureus strain Mu50, GenBank
Accession Number NP_371679 (residues 30-109):
(SEQ ID NO: 5)
QTKNVEAAKKYDQYQTNFKKQVNKKVVDAQKAVNLFKRTRTVA
THRKAQRAVNLIHFQHSYEKKKLQRQIDLVLKYNTLK;
(4) Ehp-C: S. aureus strain Mu50, GenBank
Accession Number NP_371679 (residues 41-109):
(SEQ ID NO: 6)
DQYQTNFKKQVNKKVVDAQKAVNLFKRTRTVATHRKAQRAVNLIHF
QHSYEKKKLQRQIDLVLKYNTLK

Bacterial Culture and Induction of Protein Expression. All proteins were expressed from the E. coli strain B834(DE3) and routine manipulations of bacterial cultures were carried out in accordance with standard methods. Expression cultures were grown at 37° C. in 250 ml Terrific Broth (TB) and induced at an OD₆₀₀ of 2.0 by adding IPTG to 1 mM final concentration. The cultures were incubated with vigorous shaking for an additional 18 h to achieve maximal cell density and the induced cells were harvested by centrifugation.

Bacterial Cell Lysis, Chelating Affinity Chromatography, and Protein Refolding. Pellets of induced cells were concomitantly resuspended and lysed by stirring in 5% of the original culture volume of denaturing lysis buffer (0.1 M tris (pH 8.0), 6 M guanidine-HCl). The cell pellet was stirred at 300-500 rpm for 30 min at room temperature and the solubilized, denatured proteins were separated from the cell wall and membrane debris by centrifugation (30 min at 25,000×g). Following centrifugation, the clarified cell extract was decanted and applied by gravity flow to a 2 ml column of Ni²⁺-NTA sepharose (Qiagen) that had previously been equilibrated to room temperature in denaturing wash buffer (20 mM sodium phosphate (pH 6.0), 0.5 M NaCl, 20 mM imidazole, 8 M urea). Unbound proteins and contaminants were removed from the column by applying 5 CV of denaturing wash buffer. Tagged proteins were eluted from the resin with 2.5 CV of denaturing elution buffer (20 mM sodium phosphate (pH 6.0), 0.5 M NaCl, 0.2 M imidazole, 8 M urea), though the first 0.5 CV of eluate was discarded since it contained negligible protein.

Following purification under denaturing conditions, recombinant proteins were refolded by a “rapid dilution” technique, and concentrated by native chelating chromatography. First, the denatured sample was drawn into an appropriate syringe with needle and refolded by quickly injecting the entire amount into a 10-fold volume excess of rapidly-stirring, room temperature native buffer (20 mM tris (pH 8.0), 0.5 M NaCl). Then, the diluted sample was allowed to stir for 5-10 min longer under these conditions, at which time it was reapplied to the previous column of Ni²⁺-NTA sepharose that had since been regenerated according to manufacturer's suggestions and equilibrated in native wash buffer (20 mM tris (pH 8.0), 0.5 M NaCl, 10 mM imidazole). Excess urea was removed by washing with 5 CV of native wash buffer, and the bound, refolded proteins were eluted as before with 2.5 CV of native elution buffer (20 mM tris (pH 8.0), 0.5 M NaCl, 500 mM imidazole).

Recombinant TEV protease (see above) was used to digest the fusion tag away from the target protein. Digests were carried out at 4° C. to avoid protein precipitation. Following digestion, each of the target proteins was purified further by ion exchange chromatography. In each case, the proteolytic digest was desalted into a suitable 20 mM ethanolamine buffer (pH 9.0) without salt, applied to a 6 ml Resource S or Q ion-exchange column (Amersham-Pharmacia Biotechnology), and eluted with a gradient to 1 M NaCl over 10 CV.

### Example 5

### Design of Therapeutics

The complement system plays a key role in the pathology of a continually-expanding list of inflammatory, autoimmune, and ischemic conditions. And while numerous attractive pharmacological targets in the complement cascades have been identified, there are only a limited number of anti-complement therapeutics approved for clinical use. The unique modes of C3 recognition and inhibition by both Efb and Ehp may therefore be utilized for the design and optimization of a new class of therapeutic complement inhibitors.

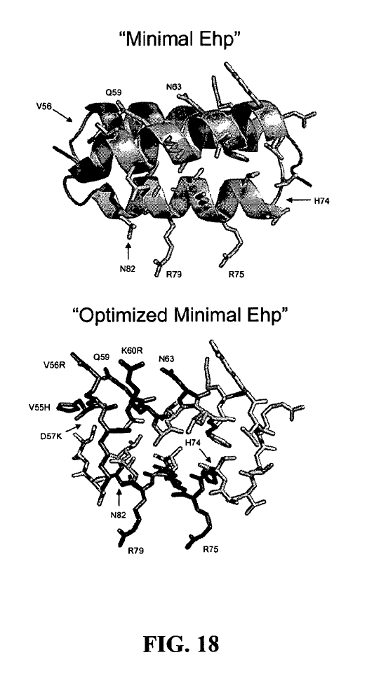

Based upon the structure/function studies described above, a simplified scheme for the design of a new class of potential complement inhibitors is shown in FIG. 18 and described in this prophetic example. To begin, Ehp is chosen as choice of a “lead compound” since it is a more potent inhibitor of the complement activation than is Efb. Next, comparison of the biochemical and structural properties of Efb and Ehp suggests that the additional inhibitory potency of the latter protein is derived from its second C3 recognition site. Examination of the Ehp sequence reveals that both C3-binding sites are contained within a minimal 29-residue stretch that comprises helices alpha 1 and alpha 2 of the Ehp protein (FIG. 18, Top Panel). The fact that the second C3-binding site in Ehp is substantially lower in affinity than the canonical Efb-like site suggests that beneficial increases in both affinity and inhibitory potency may be gained by mutating select residues so that this lower-affinity site contains residues identical to the higher-affinity site (FIG. 18, Bottom Panel).

Although the family of compounds typified by the stick representation in FIG. 18 has are then tested for complement inhibitory properties. Positive results may be expected for several reasons. First, while there are limitations to peptide-based therapeutics, the effective targeting of the alternative pathway by the peptidomimetic Compstatin suggests that this class of molecules can be utilized to successfully modulate C3 function. Second, although the number of positions in these molecules that bind directly to C3 is limited, there are still a number of ancillary positions that can be screened by combinatoric optimization to improve the complement inhibitory properties of the resulting molecules. Finally, the relatively small size of the key functional regions in these proteins suggests that effective mimics can be found in the realm of pharmacologically-available small molecules.

### REFERENCES

1. Morikis, D., and Lambris, J. D. Structural Biology of the Complement System (ed. Morikis, D., and Lambris, J. D.) (Taylor & Francis, Boca Raton, Fla., 2005).
2. Patti, J. M., Allen, B. L., McGavin, M. J., and Hook, M. MSCRAMM mediated adherence of microorganisms to host tissues. Annu Rev Microbiol 48, 585-617 (1994).
3. Chavakis, T., Hussain, M., Kanse, S. M., Peters., G., Bretzel, R. G., Flock, J.-I., Herrmann, M., and Preissner, K. T. Staphylococcus aureus extracellular adherence protein serves as anti-inflammatory factor by inhibiting the recruitment of host leukocytes. Nat. Med. 8, 687-693 (2002).
4. Lee, L. Y., Miyamoto, Y. J., McIntyre, B. W., Hook, M., McCrea, K. W., McDevitt, D., and Brown, E. L. The Staphylococcus aureus Map Protein is an Immunomodulator that Interferes with T Cell-Mediated Responses. J. Clin. Invest. 110, 1461-1471 (2002).
5. Lee, L. Y. L., Hook, M., Haviland, D., Wetsel, R. A., Yonter, E. O., Syribeys, P., Vernachio, J., and Brown, E. L. Inhibition of Complement Activation by a Secreted Staphylococcus aureus Protein. J. Infect. Dis. 190, 571-579 (2004).
6. Lee, L. Y. L., Liang, X., Hook, M., and Brown, E. L. Identification and characterization of the C3 binding domain of the Staphylococcus aureus extracellular fibrinogen-binding protein (Efb). J Biol Chem 279, 50710-50716 (2004).
7. Rooijakkers, S. H., Ruyken, M., Roos, A., Daha, M. R., Presanis, J. S., Sim, R. B., van Wamel, W. J., van Kessel, K. P., and van Strijp, J. A. Immune Evasion by a Staphylococcal Complement Inhibitor that Acts on C3 Convertases. Nat. Immunol. 6, 920-927 (2005).
8. Hornef, M. W., Wick, M. J., Rhen, M., and Normark, S. Bacterial Strategies for Overcoming Host Innate and Adaptive Immune Response. Nat. Immunol. 3, 1033-1040 (2002).
9. Sahu, A., and Lambris, J. D. Structure and Biology of Complement Protein 3, a Connecting Link Between Innate and Acquired Immunity. Immunol. Rev. 180, 35-48 (2001).
10. Neth, O., Jack, D. L., Johnson, M., Klein, N. J., and Turner, M. W. Enhancement of Complement Activation and Opsonophagocytosis by Complexes of Mannose-Binding Lectin with Mannose-Binding Lectin-Associated Serine Protease After Binding to Staphylococcus aureus. J. Immunol. 169, 4430-4436 (2002).
11. Kawasaki, A., Takada, H., Kotani, S., Inai, S., Nagaki, K., Matsumoto, M., Yokogawa, K., Kawata, S., Kusumoto, S., and Shiba, T. Activation of the Human Complement Cascade by Bacterial Cell Walls, Peptidoglycans, Water-Soluble Peptidoglycan Components, and Synthetic Muramylpeptides—Studies on Active Components and Structural Requirements. Microbiol. Immunol. 31, 551-569 (1987).
12. Bredius, R. G., Driedijk, P.C., Schouten, M. F., Weening, R. S., and Out, T. A. Compelment Activation by Polyclonal Immunoglobulin G1 and G2 Antibodies Against Staphylococcus aureus, Haemophilus influenzae type B, and Tetanus Toxoid. Infect. Immun. 60, 4838-4847 (1992).
13. Verbrugh, H. A., Van Dijk, W. C., Peters, R., Van Der Tol, M. E., and Verhoef, J. The Role of Staphylococcus aureus Cell-Wall Peptidoglycan, Teichoic Acid, and Protein A in the Processes of Complement Activation and Opsonization. Immunology 37, 615-621 (1979).
14. Wilkinson, B. J., Kim, Y., Peterson, P. K., Quie, P. G., and Michael, A. F. Activation of Complement by Cell Surface Components of Staphylococcus aureus. Infect. Immun. 20, 388-392 (1978).
15. Sakiniene, E., Bremell, T., and Tarkowski, A. Complement Depletion Aggravates Staphylococcus aureus Septicaemia and Septic Arthritis. Clin. Exp. Immunol. 115, 95-102 (1999).
16. Palma, M., Nozohoor, S., Schennings, T., Heimdahl, A., and Flock, J. -I. Lack of the Extracellular 19-Kilodalton Fibrinogen-Binding Protein from Staphylococcus aureus Decreases Virulence in Experimental Wound Infection. Infect. Immun. 64, 5284-5289 (1996).
17. Nagar, B., Jones, R. G., Diefenbach, R. J., Isenman, D. E., and Rini, J. M. X-ray Crystal Structure of C3d: A C3 Fragment and Ligand for Complement Receptor 2. Science 280, 1277-1281 (1998).
18. Janssen, B. J. C., Huizinga, E. G., Raaijmakers, H. C. A., Roos, A., Daha, M. R., Ekdahl-Nilsson, K., Nilsson, B., and Gros, P. Structures of Complement Component C3 Provide Insights Into the Function and Evolution of Immunity. Nature 437, 505-511 (2005).
19. Janssen, B. J. C., Christodoulidou, A., McCarthy, A., Lambris, J. D., and Gros, P. Structure of C3b Reveals Conformational Changes Underlying Complement Activity. Nature, In Press (2006).
20. Isenman, D. E., and Cooper, N. R. The Structure and Function of the Third Component of Human Complement 1. The Nature and Extent of Conformational Changes Accompanying C3 Activation. Mol. Immunol. 18, 331-339 (1981).
21. Isenman, D. E., Kells, D. I. C., Cooper, N. R., Muller-Eberhard, H. J., and Pangburn, M. K. Nucleophilic Modification of Human Complement Component Protein C3: Correlation of Conformational Changes with Acquisition of C3b-like Functional Properties. Biochemistry 20, 4458-4467 (1981).
22. Isenman, D. E. Conformational Changes Accompanying Proteolytic Cleavage of Human Complement Protein C3b by the Regulatory Enzyme Factor I and its Cofactor H. Spectroscopic and Enzymological Studies. J. Biol. Chem. 258, 4238-4244 (1983).
23. Nilsson, B., Grossberger, D., Nilsson-Ekdahl, K., Riegert, P., Becherer, D., and Lambris, J. D. Confomational Differences Between Surface-bound and Fluid-phase Complement Component-C3 Fragments. Epitope Mapping by cDNA Expression. Biochem. J. 282, 715-721 (1992).
24. Lindahl, G., Sjobring, U., and Johnsson, E. Human Complement Regulators: a Major Target for Pathogenic Microorganisms. Curr. Opin. Immunol. 12, 44-51 (2000).
25. Geisbrecht, B. V., Bouyain, S., and Pop, M. An Optimized System for the Expression and Purification of Secreted Bacterial Proteins. Prot. Expr. Purif. 46, 23-32 (2006).
26. Guthridge, J. M., Rakstang, J. K., Young, K. A., Hinshelwood, J., Aslam, M., Robertson, A., Gipson, M. G., Sarrias, M.-R., Moore, W. T., Meagher, M., Karp, D., Lambris, J. D., Perkins, S. J., and Holers, V. M. Structural Studies in Solution of the Recombinant N-Terminal Pair of Short Consensus/Complement Repeat Domains of Complement Receptor Type 2 (CR2/CD21) and Interactions with Its Ligand C3dg. Biochemistry 40, 5931-5941 (2001).
27. Hammel, M., Ramyar, K. X., Spencer, C. T., and Geisbrecht, B. V. Crystallization and X-ray Diffraction Analysis of the Complement Component-3 (C3) Inhibitory Domain of Efb from Staphylococcus aureus. Acta. Cryst. F. 62, 285-288 (2006).
28. Terwilliger, T. C., Berendzen, J. Automated MAD and MIR structure solution. Acta Cryst. D. 55, 849-861 (1999).
29. Terwilliger, T. C. Maximum likelihood density modification. Acta Cryst. D. 56, 965-972 (2000).
30. Terwilliger, T. C. Automated main-chain model-building by template-matching and interative fragment extension. Acta Cryst. D. 59, 34-44 (2002).
31. Jones, T. A., Zou, J.-Y., Cowan, S. W., and Kjeldgaard, M. Improved methods for the building of protein models in electron density maps and the location of errors in the models. Acta Cryst. D. 47, 110-119 (1991).
32. Brunger, A. T., Adams, P. D., Clore, G. M., DeLano, W. L., Gros, P., Grosse-Kunstleve, R. W., Jiang, J.-S., Kuszewski, J., Nilges, M., Pannu, N. S., Read, R. J., Rice, L. M., Simonson, T., and Warren, G. L. Crystallography & NMR System: A New Software Suite for Macromolecular Structure Determination. Acta Cryst. D. 54, 905-921 (1998).
33. Murshudov, G. N., Lebedev, A., Vagin, A. A., Wilson, K. S., and Dodson, E. J. Efficient Anisotropic Refinement of Macromolecular Structures Using FFT. Acta. Cryst. D. 55, 247-255 (1999).
34. Winn, M., Isupov, M., and Murshudov, G. N. Use of TLS Parameters to Model Anisotropic Displacments in Macromolecular Refinement. Acta. Cryst. D. 57, 122-133 (2001).
35. The Collaborative Computational Crystallography Project 4. The CCP4 Suite: Programs for Protein Crystallography. Acta Cryst. D. 50, 760-763 (1994).
36. Nicholls, A., Sharp, K. A., and Honig, B. Protein Folding and Association: Insights from the Interfacial and Thermodynamic Properties of Hydrocarbons. Proteins 11, 281-296 (1991).
37. Zemla, A. LGA: a method for finding 3D similarities in protein structures. Nucleic Acids Research 31, 3370-3374 (2003).
38. Kraus, D., Medof, D. E., and Mold, C. Complementary Recognition of Alternative Pathway Activators by Decay-Accelerating Factor and Factor H. Infect. Immun. 66, 399-405 (1998).
39. Sfyroera, G., Katragadda, M., Morikis, D., Isaacs, S. N., and Lambris, J. D. Electrostatic Modeling Predicts the Activities of Orthopoxvirus Complement Control Proteins. J. Immunol. 174, 2143-2151 (2005).
40. Becherer, J. D., Alsenz, J., Esparza, I., Hack, C. E., and Lambris, J. D. A Segment Spanning Residues 727-768 of the Complement C3 Sequence Contains a Neoantigenic Site and Accommodates the Binding of CR1, Factor H, and Factor B. Biochemistry 31, 1787-1794 (1992).
41. Myszka, D. G., and Morton, T. A. CLAMP: a Biosensor Kinetic Data Analysis Program. Trends Biochem. Sci. 23, 149-150 (1998).

The present invention is not limited to the embodiments described and exemplified above, but is capable of variation and modification within the scope of the appended claims.

What is claimed:

1. A peptide comprising a sequence selected from:
(SEQ ID NO: 1)
KKEQKLIQAQNLVREFEKTHTVSAHRKAQKAVNLVSFEYKVKKMVLQERI
DNVLKQGLVR
or
(SEQ ID NO: 2)
KKVVDAQKAVNLFKRTRTVATHRKAQRAVNLIHFQHSYEKKKLQRQIDLV
LKYNTLK;
wherein the peptide has a folded structure comprising, in order from the N- to C-terminus, a first α1 helix of about 20 residues, a second α2 helix of about 13 residues, and a third α helix of about 17 residues, and comprising a random coil conformation of about 4 residues at its C-terminus, wherein the first and third α helix are substantially parallel and the second α helix is substantially anti-parallel to the first and third α helices, and wherein the peptide binds to C3, induces a conformational change in C3, and inhibits C3-mediated complement pathway activation.

2. The peptide of claim 1, comprising a structure substantially the same as a structure of a peptide shown in FIG. 1 for a C-terminal portion of Efb.

3. The peptide of claim 1, comprising a structure substantially the same as a structure of a peptide as shown in FIG. 2, for a C-terminal portion of Efb, bound to C3.

4. The peptide of claim 1, which is a C-terminal portion of Efb.

5. The peptide of claim 1, contained within Ehp.

6. An analog of the peptide of claim 1 that retains the structure and function of the peptide, optionally comprising one or more of (1) naturally-occurring amino acids, (2) non-naturally-occurring amino acids, or (3) compounds that are not amino acids.

7. A polynucleotide that encodes the peptide of claim 1.

8. An antibody immunologically specific for the peptide of claim 1, or for a binding region on C3 to which the peptide of claim 1 binds.

9. A method of inhibiting complement activation, comprising contacting a complement-forming system with the peptide of claim 1, under conditions permitting binding of the peptide to C3, resulting in the inhibition of the complement activation.

10. The method of claim 9, wherein the complement-forming system is contained within a living organism.

11. A complex comprising C3 and a polypeptide secreted from Staphylococcus aureus, comprising SEQ ID NO:2, wherein the C3 within the complex is incapable of activating a complement pathway.

12. The complex of claim 11, wherein the polypeptide is SAV1155, GenBank Accession Number NP 371679.

13. A method of inhibiting complement activation, comprising contacting a complement-forming system with a polypeptide secreted from Staphylococcus aureus that comprises SEQ ID NO:2, under conditions permitting binding of the peptide to C3, resulting in the inhibition of the complement activation.

14. The method of claim 13, wherein the polypeptide is SAV1155, GenBank Accession Number NP 371679.

15. The method of claim 13, wherein the complement-forming system is contained within a living organism.
